(19) United States
(12) Patent Application Publication
(10) Pub. No.: US 2020/0390568 A1
(43) Pub. Date: Dec. 17, 2020

(54) PROSTHETIC LIMB SOCKETS AND METHODS OF MAKING AND USING

(71) Applicant: MEDICAL CREATIONS, INC., Aspen, CO (US)

(72) Inventor: Mark Joseph, Aspen, CO (US)

(21) Appl. No.: 17/001,380

(22) Filed: Aug. 24, 2020

Related U.S. Application Data
(63) Continuation-in-part of application No. 15/914,480, filed on Mar. 7, 2018, now Pat. No. 10,751,202.

(51) Int. Cl. A61F 2/80; A61F 2/50; B29C 45/13

(52) U.S. Cl. CPC A61F 2/80; A61F 2/5046; A61F 2002/5053; A61F 2002/5052; B29C 45/13

(57) ABSTRACT

A prosthetic socket includes a conical cup, an outer layer on the outer surface of the conical cup, and a reinforcement layer on the inner surface of the conical cup. The prosthetic socket is shapeable after being heated to a shaping temperature. The outer layer is less malleable than the conical cup at the shaping temperature, but has a higher rigidity than the conical cup at the shaping temperature and has smoother outer surface than an outer surface of the conical cup. The reinforcement layer has a higher resistance against circumferential stress than the conical cup. A residual limb or a model of a residual limb can be inserted into the preformed prosthetic socket. The prosthetic socket is then heated to the shaping temperature and molded to conform to the contour of the residual limb or the model to form a prosthetic socket.

### CROSS-REFERENCE TO RELATED APPLICATIONS

The present application is a continuation-in-part of U.S. patent application Ser. No. 15/914,480, filed Mar. 7, 2018, the above-identified application is fully incorporated herein by reference.

### BACKGROUND OF THE INVENTION

### Field of the Invention

The present disclosure generally relates to medical devices, prosthetic devices, sockets for prosthetic limbs, and more specifically, to prosthetic sockets having improved conformability to a patient's residual limbs.

### Discussion of the Related Art

A prosthetic socket for amputated limbs typically fits precisely and tightly to the residual limb to bear the weight of a patient previously placed on the now-missing limb, spreading the force from amputated bone ends and bone prominences and to soft portions of the residual limb. Fitting prosthetic sockets to residual limbs is carried out by an experienced prosthetist (as a prosthetic practitioner is called), and typically requires a high degree of training and experience. A related art process for making such sockets requires casting of the residual limb by wrapping with casting plaster (similar to making a fracture cast) to make a negative form, then filling the form with plaster to make a precise positive model of the residual limb.

The prosthetist must carefully evaluate the residual limb, alignment, stance and sensitivities, and determine the desired load bearing characteristics of the socket. Typically, to achieve a proper tight fit, the entire model must be reduced in circumference to some degree. In some areas, the model may be increased, or material added to create pockets that reduce contact with sensitive areas of the residual limb. Using abrasive files, sandpaper, and scraping tools, the prosthetist adjusts the diameter and shape of the model by hand, in an imprecise manner, to approximate the shape that is presumed to be appropriate for the intended outcome. Some prosthetists use computer scanning and manipulation to achieve this, but that process is still experience based and imprecise.

Once the model shaping process is complete, the socket is created by heating high temperature thermoplastics to temperatures, e.g., above 320° F., and forming them over the model using complicated and time-consuming techniques. Typically, over half of the materials required become scrap and must be disposed of. In many cases, the socket is made from fiberglass and toxic two-part resin. The resin must be cured, and forms a very rigid socket that can only be modified by grinding away material. These sockets are finished on the edges and surfaces using time-consuming and messy methods, and are then test fitted to the residual limb. Because the model shaping process is imprecise and based on estimation, often several time-consuming adjustments must be made to the socket for proper fitment, requiring several visits to the prosthetist and time consuming techniques to make adjustments.

This entire related art process is complicated, time consuming and requires a large workshop with expensive machinery, ventilation, and extensive materials inventory. Many prosthetists perform only the initial casting and final fitting procedures because they do not have a sufficient workshop, and must send away to a socket making service further complicating the process and adding cost and time. Often, it is determined that the socket was improperly made and the entire process must be started again.

In the case of recent amputees, the residual limb is very sensitive, and over the period of months, can atrophy and shrink substantially, change shape, and develop callus. During this time, temporary “test” sockets are made using the above process, yet frequently with less durable materials because the sockets may only be worn for a short period until a subsequent one, started from scratch, is needed. This process may be repeated from three to five times depending on the amputation and amputee conditions, thereby significantly increasing the time, effort, waste, office visits, travel and efforts required of all involved.

This process is very stressful for the amputee. It is painful, time consuming, and often requires distant travel. It can take hours and days of waiting for adjustments to be made. The amputee typically must make several trips to the prosthetist and wait days or weeks for the socket to be completed. The process of making adjustment is limited, and therefore the entire process must be repeated if the prosthetist is unable to adjust the socket enough to achieve the desired results. Then, the amputee must get used to wearing the new socket which can involve weeks of pain, the final outcome being unknown until comfort is achieved.

Cost is another serious consideration. Insurance, which may cover prosthetics, can be extremely limited, and may not pay for another prosthetic for years, even if the current one is working poorly. For the prosthetist, insurance reimbursement is often a one-time fee based on the amputation and equipment approved. The number of times the prosthetic must be made and adjusted is not reimbursed for, and therefore the prosthetist loses profit every time the patient returns. The process is so difficult that amputees often put up with less than desirable fit, chronic pain, and use/walking challenges for months or even years before going through the process again.

In order to improve upon the challenges of making a socket in the conventional manner previously described, attempts have been made in the past to direct mold low temperature thermoplastic sockets onto residual limbs. These methods have not been widely accepted or used by prosthetists. Low temperature thermoplastic materials, such as polycaprolactone, have been used. Such related art materials are formable at between 120° F. and 180° F. (50° C. to 71° C.). They are typically heated in hot water and can be applied directly to the skin. These materials have inadequate strength and rigidity to hold up to the rigors of weight bearing and the abuse of walking. They also tend to become very difficult to work with because when heated they become clay-like, extremely sticky and are very difficult to form tightly to the limb. Therefore, the sockets produced using these materials are inadequate and undesirable. While these sockets may occasionally be used as temporary sockets, they are typically not used as permanent sockets.

Other currently-used sockets have a hard supportive outer shell made in the typical fashion, and a softer, low temperature direct on-body heat-formed inner liner. While these sockets can perform adequately, they still require the same time-consuming steps needed to make the outer socket which is rigid, made of high temperature thermoplastics or fiberglass, and must be custom made using casting and a model as described above. Additional steps are required to mold the inner soft non-supportive liner using a heat forming direct-on-body process. If the outer hard socket is not properly formed and fitted, the inner soft liner may not adequately adjust to the residual limb.

Forming temperatures above 300° F. are impractical because the residual limb would be burned by such hot materials applied to the body, even with an insulative liner. It should be noted that the physical properties of the related devices made of related art polymers thermoformed under 325° F. (about 163° C.) lack durability, have poor elongation, have poor crack resistance and rigidity. Sockets made of these materials are extremely difficult to remold and adjust.

The previously described methods of directly heat forming sockets to residual limbs have been largely unsuccessful and, as a result, are not prevalent in the market.

### SUMMARY

The present disclosure describes prosthetic sockets also referred herein as prosthetic limb sockets for amputated limbs that are formed directly to the residual limb of the patient using proprietary materials that are dry-heated to become formable, pliable and stretchable. This eliminates the step of casting the residual limb along with the model making process, and drastically reduces the number of steps required to make a socket. Direct forming also reduces the imprecise hand grinding and shaping method currently used to adjust the model, in addition to forming the final high temperature plastic or fiberglass/resin socket to the model. Sockets in accordance with the present disclosure utilize the residual limb to form the socket and create a precise, tight fit. The present disclosure describes original features, materials and methods that will allow prosthetists to quickly and efficiently direct-heat form a prosthetic socket to the residual limb or model which has a precise tight fit, is durable, light weight, reformable, and saves time, materials, and cost.

Sockets in accordance with the present disclosure are formed from proprietary thermoplastic materials which allow the sockets to be heat formed or molded at temperatures in a range from about 160° F. to about 305° F. and any sub-range in between, in a preferred embodiment in a temperature in a range from about 225° F. to 290° F., and in more preferred embodiment a temperature in a range from about 250° F. to about 285° F. A prosthetic liner sufficiently thick and insulative to protect the residual limb from the higher temperatures is worn on the limb during forming of the socket. Amputees commonly wear such liners to protect the residual limb and hold the socket firmly to it. They can, for example, comprise stretchable gel with an outer stretch fabric lining. The gel may comprise silicone, polyurethane or other similar materials that are compatible with the skin and, using circumferential tightness, will hold firmly to the limb. Various attachment means may be used to hold the liner to the socket, and sockets in accordance with the present disclosure may be compatible with such attachment means. Other suitable insulation types may include fabric liners comprising cotton, various foams, and other materials that are sufficiently insulative.

Thermoplastic materials that form at higher temperatures can be engineered chemically to have improved physical properties over those used in previous lower temperature methods, such as those methods as previously described herein. Leg prosthetics, for example, are subjected to many thousands of steps, high body weight, running, jumping and other actions, which exert considerable force on the socket and to its attachment to the replacement leg prosthetic. Arm prosthetics, while not bearing weight, can also require very strong construction. Embodiments of prosthetic sockets herein have excellent properties including but not limited to excellent rigidity properties, excellent stiffness properties, excellent impact strength properties, excellent elongation properties, excellent impact resistance properties, excellent resistance to crack propagation and creep properties .

In one embodiment, methods of making sockets directly on a residual limb using thermoplastics that become malleable, stretchable, and formable at higher temperature ranges, such as, for example, a temperature in a range from about 160° F. to about 305° F. and any sub-range in between, in a preferred embodiment in a temperature in a range from 225° F. to 290° F., and in more preferred embodiment a temperature in a range from 250° F. to about 285° F. This temperature range is novel and ideal because it is the highest range that can be comfortably and safely formed over the residual limb with an insulating gel liner as previously described. It is also a considerably higher temperature than previous attempts have used and therefore, the thermoplastic can have adequate properties of rigidity, elongation, impact resistance, crack propagation and creep in order to be adequately durable. Optionally and/or alternatively, various additives can be incorporated into the higher temperature thermoplastic to improve strength including but not limited to carbon fiber, aramid fiber, fiberglass, glass micro beads, carbon nanotubes beads, and others.

In one embodiment, sockets are formed by injection molding. A number of suitable sizes and shapes are provided which correspond to the various common residual limb sizes. When a socket is sized by the prosthetist, it will be smaller in circumference than the residual limb so that it can be stretched on for a tight fit. In various embodiments, sockets may be injection molded of a single polymer type, or a secondary over molding process can be used to co-join two different polymers to provide varied featured in various places. For example, the upper portion of a socket can be made of a polymer that becomes more formable in the target temperature range so that it can be easily formed to the limb details.

In one embodiment, a lower portion of a socket comprises a polymer, e.g., thermoplastic material, that is less formable than the upper portion in the target heat range. This lower portion can, when heated, provide a more supportive area for a socket base as the upper portion is stretched over the limb, and the limb is forced down into the socket. However, it can also conform to the residual limb to some degree. The lower portion can also be a material that has a higher forming temperature and thus can have even greater physical properties to accommodate the higher forces applied to the base of the socket and the attachment to the prosthetic. The lower portion is also required to hold its shape when heated so that the connections to the prosthetic, and other critical features, do not deform during the heating and forming processes. Additionally, the socket can comprise a third polymer section at the base that does not become malleable at the target temperature range in order to hold its features precisely during heating and forming. This portion may be over molded as well, or attached by fasteners to the upper socket section.

In various embodiments, complex and detailed features can be molded into the socket to achieve various effects by, for example, injection molding. The polymer materials can be varied in either composition or thickness to precisely provide increased support or more formability as desired. Three dimensional elements can be used to provide support and strength such as ribbing, tapered sections, corrugations, cross hatched reinforcements and the like. For below-the-knee amputations, for instance, areas near the end of the tibia and fibula can require extra care in forming so that the socket provides extra room in these areas. These areas could be made thinner and thus easier to expand and form. Adjacent areas could have ribbing to provide more support and strength.

Once the socket is formed to the amputee, the top can be trimmed and smoothly finished. This can be efficiently performed by heating the top portion with a heat gun and using shears to cut the top to the preferred height and shape. A hand rotary electric grinder can be used to smooth the top edge. Spot heat can be applied to adjust the top edge, to flair portions and tighten others to the limb. These tools can be portable and may produce only a small amount of grit and dust that can be easily contained and cleaned up.

At any time, due to the low temperature formability of the polymer, a forced air heat gun can be used to spot heat the socket, often while the socket is worn by the amputee. Pressure can be applied, and adjustments quickly made in a precise manner. Additionally, if the socket shape proves dysfunctional, the entire socket or a portion of the socket can be reheated and reformed quickly and precisely. This can prove especially valuable in the case of atrophy or changes to the muscles over time. Rather than starting the typical process over again by discarding the socket and casting a new one, this reheating and reforming process saves a great deal of time, materials, and expense. The time required to form and finish a socket of the present disclosure may, for example, be under two hours, and can be completed in one sitting. Compare this to the many hours, steps, drying and curing time, finishing, and adjusting time required for the typical socket, which can take days or weeks. The benefits to the prosthetist and amputee are considerable.

In one embodiment, a process of making a socket requires a great deal of attention be paid to the base of the socket where the locking mechanism for the socket connects to the gel liner. Also, the metal connection to the leg or arm extension must be built into the base in a strong manner. Typically, a large inventory of the various locking mechanisms and attachment parts must be maintained to meet the various needs of the prosthetic. The prosthetist hand builds these devices into the base of the socket requiring a great deal of skill, knowledge and time. Additionally, the suspension system can be built into the socket permanently so they are not interchangeable and the system must be decided on before building the socket. This prevents the patient from trying different systems to see which one works best for them. In various embodiments, injection molding of a socket permits the addition of elements or physical features into the socket, such as the base, adjustment, and locking mechanisms. For example, elements used by a locking mechanism of a prosthetic can be modular, interchangeable, and insert quickly into the base of the socket so the prosthetist can simply pick the type of locking mechanism and attach any number of connectors for the leg extension in minutes. These can all be quickly changed at any time so the patient and practitioner can experiment with different systems to determine the best one. In some cases, the patient themselves can interchange systems, for example, pin lock suspension for one purpose, and a suction system for another.

In one embodiment, the injection molding process is a precision process that can also allow the base of the socket to include adjustment mechanisms for attaching the prosthetic. Often, the prosthetic must be offset horizontally from the center of the bottom of the socket in order to properly align the socket for optimal use, gait, and balance. Sockets can, for example, include an attachment member for the prosthetic that uses the common four flat head bolts typically used to attach the metal base plate. This base plate attaches in an angularly adjustable manner to the prosthetic. By loosening the four bolts, the base plate can be slid and adjusted in a planar horizontal manner to offset the prosthetic as desired. Tightening the four bolts locks it in place. This feature complements the easy adjustability of the heat formable socket by providing instant alignment to save the amputee and prosthetist time, materials and cost. Additionally, angular adjustment can be achieved by a ball and socket type of connection to the lower base that can be loosened, rotated, and tightened.

Methods of the forming sockets of the present disclosure may significantly reduce the equipment required to fit and finish a socket. For example, a heating source, e.g. a heating bag, less than 2 inch by 1 inch by 6 inch is simply plugged in and can be safely handled due to its insulated nature. Further, a hand-held heat gun is small, inexpensive, and portable. The tools required by the methods of the present disclosure can include gloves, elastic straps, vacuum bags and the like, which are relatively small and portable. The equipment required to cut and finish the top of the socket is again, handheld, plugged in and portable. All of the tools required to fit and finish the invention of the exemplary embodiment can be fitted into a suitcase making the system portable, non-toxic, and relatively mess free. This allows for prosthetists in small offices, hospitals, and clinics to fit and finish sockets themselves in under two hours. This system can also be mobile, so that prosthetists could make house and hospital calls to provide finished sockets. The potential in rural areas and developing countries is enormous.

Sockets made in accordance with the present disclosure significantly improve the efficiency and outcome of making an amputee prosthetic socket, and allow for quick adjustment and reforming to achieve the best possible fit, comfort, and function.

Prosthetic limb sockets in accordance with the present disclosure can comprise a conical cup comprising a material having a first pliability in a temperature range from about 160° F. to about 305° F. and any sub-range in between, in a preferred embodiment the temperature range is from 225° F. to 290° F., and in a more preferred embodiment the temperature range is from 250° F. to about 285° F. The conical cup at this temperature can be stretched circumferentially over a residual limb, a lower portion coupled to a lower surface of the conical cup creating an enclosed form having a second pliability which is less than the first pliability, and a base coupled to the lower portion, wherein the conical cup and the lower portion are injection molded of a thermoplastic polymer, wherein the conical cup and the lower portion, when heated to between about 160° F. and about 305° F. (between about 70° C. and about 150 ° C.) have a working time of between about five minutes and about 15 minutes before hardening as room temperature is approached, and wherein the conical cup and the lower portion each comprise a hardness exceeding ASTM D2240 of 50D shore hardness, a tensile strength exceeding ASTM D638 of 5,000 psi, and a flexural modulus exceeding ASTM D5023 of 150,000 PSI.

The conical cup and the lower portion can be unitary. Further, at least one of the conical cup and the lower portion can be injection molded, and the other of the conical cup and the lower is over-molded. The thermoplastic polymer of the conical cup can comprise at least one additive from the group of fiberglass, carbon fiber, aramid fiber, glass beads, and carbon nanotubes. The base can comprise a securing element.

The socket can further include an insert layer including one of a rubber, a polyurethane, an Estane®, spandex, a long chain polymer. The insert layer can be insert molded into the conical cup, and can cause the heated conical cup to become elastic and draw tight circumferentially over the residual limb as it is applied and formed. The socket can further comprise a thin outer layer surrounding at least a portion of the conical cup, and the outer layer can be co-molded or adhered to an external surface of the socket. Such an outer layer can provide body and support to the heated socket and be colored, printed, or decorated. The socket can further comprise an insulating layer attached to the residual limb and secured within the conical cup.

### BRIEF DESCRIPTION OF THE DRAWINGS

The features and advantages of the present disclosure will become more apparent from the detailed description set forth below when taken in conjunction with the drawings, wherein:

FIG. 1 illustrates a side view of a prosthetic limb socket in accordance with the present disclosure;

FIG. 2 illustrates a perspective view of the prosthetic limb socket of FIG. 1 in accordance with the present disclosure;

FIG. 3 illustrates an exploded view of the prosthetic limb socket of FIGS. 1 and 2 in accordance with the present disclosure;

FIG. 4 illustrates another perspective view of the prosthetic limb socket of FIGS. 1-3 in accordance with the present disclosure;

FIG. 5 illustrates a perspective view of the prosthetic limb socket of FIGS. 1-4 in an offset configuration in accordance with the present disclosure;

FIG. 6 illustrates a cut-away side view of the prosthetic limb socket of FIGS. 1-5 in accordance with the present disclosure;

FIG. 7 illustrates a side cut-away view of another prosthetic limb socket in accordance with the present disclosure;

FIG. 8 illustrates a side cut-away view of yet another prosthetic limb socket in accordance with the present disclosure;

FIG. 9 illustrates a side view of a prosthetic limb socket in accordance with the present disclosure;

FIG. 10 illustrates an exploded view of the prosthetic limb socket of FIG. 9, in accordance with the present disclosure;

FIG. 11 illustrates a side view of the prosthetic limb socket of FIGS. 9 and 10 in an offset configuration in accordance with the present disclosure;

FIG. 12 illustrates a perspective view of the prosthetic limb socket of FIGS. 9 and 10 in an offset configuration in accordance with the present disclosure;

FIG. 13 illustrates a side cut-away view of the prosthetic limb socket of FIGS. 9 and 10 in an offset configuration in accordance with the present disclosure; and

FIG. 14 illustrates a perspective view of a prosthetic limb socket and an insulating cover in accordance with the present disclosure.

FIG. 15A is a perspective view of a prosthetic socket in accordance with an embodiment.

FIG. 15B is a perspective view of a prosthetic socket of FIG. 15A after being heated in accordance with an embodiment.

FIG. 16A is a perspective view of a prosthetic socket in accordance with an embodiment.

FIG. 16B is a perspective view of a prosthetic socket of FIG. 16A after being heated in accordance with an embodiment.

FIG. 16C is a cross-sectional view of FIG. 16A.

FIG. 16D is a perspective view of a prosthetic socket in accordance with an embodiment with an outer layer.

FIG. 17A is a top view of an outer layer configured to be attached to a conical cup of a prosthetic socket according to an embodiment.

FIG. 17B is a top view of an outer layer configured to be attached to a lower portion prosthetic socket according to an embodiment.

FIG. 18A is a top view of an outer layer configured to be attached to a conical cup of a prosthetic socket according to an embodiment.

FIG. 18B is a side view of the outer layer of FIG. 18A in a second orientation.

FIG. 19A is a perspective view of a prosthetic socket in accordance with an embodiment without an outer layer.

FIG. 19B is a perspective view of the prosthetic socket of FIG. 19A in accordance with an outer layer partially arranged over a portion of the prosthetic socket.

FIG. 19C is a perspective view of the prosthetic socket of FIG. 19A in accordance with the outer layer applied to the prosthetic socket.

FIG. 19D is a perspective view of the prosthetic socket of FIG. 19A in accordance with the outer layer applied to the prosthetic socket in a trimmed configuration.

FIG. 20 is a flow chart illustrating a process of forming a prosthetic socket, in accordance with an embodiment.

FIG. 21A is a perspective view of a prosthetic socket in accordance with an embodiment.

FIG. 21B is a perspective view of a mold for a prosthetic socket in accordance with an embodiment.

FIG. 21C is a perspective view of a mold for a prosthetic socket and a prosthetic socket in accordance with an embodiment.

FIG. 21D is a perspective view of a molded prosthetic socket in accordance with an embodiment.

FIG. 21E is a perspective view of a trimmed molded prosthetic socket in accordance with an embodiment.

FIG. 22A is a perspective view of a prosthetic socket in accordance with an embodiment.

FIG. 22B is a perspective view of a partial cross-section prosthetic socket in accordance with FIG. 20A.

### DETAILED DESCRIPTION OF THE ILLUSTRATED EMBODIMENTS

Persons skilled in the art will readily appreciate that various aspects of the present disclosure can be realized by any number of methods and articles configured to perform the intended functions. Stated differently, other methods and articles can be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

Prosthetic limb sockets in accordance with the present disclosure are used to secure prosthetic limbs to the residual limb of a patient. In many cases, the prosthetist and patient select an appropriate liner to apply to the residual limb. The liner reduces discomfort (such as chafing or rubbing) between the skin of the residual limb and a socket. The liner also has a very high friction interior that adheres to the skin to hold it in place during movement and has a connection means to the socket that can vary. The socket is applied over the liner, and acts to support and suspend a prosthetic limb to the residual limb of the patient. Stated another way, the liner is positioned between a residual limb and a socket, and the actual prosthetic limb is coupled to the socket.

In various embodiments, sockets comprise an upper portion and a lower portion. In certain embodiments, the upper portion has a first pliability in a given temperature range which is greater than the pliability of the lower portion in the same temperature range. In one embodiment, the lower portion serves to support the socket during heat forming yet is still conformable when heated. The lower portion also has means to attach the prosthetic limb in an adjustable fashion and has attachment member for various mechanisms to lock the gel liner to the socket. In this case, the lowest portion that performs this function is not heated so it retains its shape and mechanical properties.

In yet other embodiments, the lower portion comprises a middle portion and a base, the base comprising a polymer that does not become malleable at all in the same temperature range that the upper portion and the middle potion become malleable. The upper portion interacts with and surrounds the residual limb (including, in most cases, a liner). The middle portion is co-molded to the upper portion and serves to support the socket during heat forming, yet is still conformable when heated, and is attached mechanically to the base. The base is adjustable in alignment and acts as an attachment portion for the prosthetic limb, coupling it to the socket (and in turn, to the residual limb of the patient). Sockets in accordance with the present disclosure comprise upper portions (referred to as conical cups) having improved flexibility, comfort, and/or engagement with the residual limb. In yet other embodiments, the socket is made of a single material that is pliable when heated, similar to the upper portion previously described. It is heated in a fashion so that the upper portion is heated more than the lower portion. In this manner, the upper portion will be more pliable, the middle portion will be moderately pliable, and the base will remain rigid during the forming process so as to retain its mechanical shape and properties. In some embodiments, the conical cup can be fabricated by blow molding, injection material or other techniques.

In one embodiment, the conical cup is formed from a thermoplastic or polymeric material, e.g., a polyester material, a polyester blend, and a polyester blend with one or more additives. In one embodiment, the one more additives include a carbon fiber additive to increase strength and modulus and another additive to the lower the forming temperature and widen the glass transition range to increase working time.

In some embodiments, the outer layer includes a polymer material that becomes pliable or moldable at a shaping temperature, e.g., a thermoplastic material, a polyethylene terephthalate (PET) material, a polyester material, polyvinyl chloride (PVC) material, and combinations of the same.

The shaping temperature is a temperature where the conical cup or the outer layer become pliable and stretchable, e.g., a temperature in range from about 160° F. to about 305° F. and any sub-range in between, in a preferred embodiment a temperature in a range from about 225° F. to 290° F., and in more preferred embodiment a temperature in a range from about 250° F. to about 285° F.

In one embodiment, an outer layer for a prosthetic socket includes an outer layer configured to be arranged around an outer circumference of a conical cup of the prosthetic socket and configured to be adhered to an outer surface of the conical cup of the prosthetic socket during a co-injection molding process. The conical cup of the prosthetic socket includes a first end having an opening configured to receive at least a portion of a residual limb of a user and a second end being substantially closed. The outer layer includes a thermoplastic material and has a thickness in range from of about 0.1 mm to about 1 mm. The outer layer thermoplastic material is malleable after being heated to a shaping temperature and the thermoplastic material is less malleable than a material of the conical cup of the prosthetic socket at the shaping temperature and at least a portion of the conical cup of the prosthetic socket is configured to be contained within the outer layer. The outer layer and the conical cup of the prosthetic socket are configured to be substantially simultaneously formed to one or more contours of a residual limb or one or more contours of a model of a residual limb at the shaping temperature. Also, during the forming the outer layer is less malleable than the conical cup of the prosthetic socket at the shaping temperature and the outer layer includes a smoother outer surface as compared to an outer surface of the conical cup of the prosthetic socket at room temperature. The outer layer has a higher abrasion resistance and scratch resistance as compared to an outer surface of the conical cup of the prosthetic socket at room temperature.

In one embodiment, the outer layer for a prosthetic socket includes a pre-dimensioned outer layer configured to be co-injected to form a prosthetic socket having a conical cup, a lower portion and the pre-dimensioned outer layer attached to an outer circumference of the conical cup. The pre-dimensioned outer layer includes an inside surface, an opposite outer surface, a top, a bottom, a first side extending from the bottom to the top a first angle measured between the first side and the bottom, and second side opposite the first side extending from the bottom to the top at a second angle measured between the second side and the bottom, and a thickness in range from of about 0.1 mm to about 1 mm. The pre-dimensioned outer layer includes a thermoplastic material that is malleable after being heated to a shaping temperature. The pre-dimensioned outer layer and the conical cup of the prosthetic socket are configured to be substantially mimic one or more contours of a residual limb or one or more contours of a model of a residual limb at the shaping temperature. The outer layer and the conical cup of the prosthetic socket are configured to be substantially simultaneously formed to substantially mimic one or more contours of a residual limb or one or more contours of a model of a residual limb at the shaping temperature.

One embodiment is directed towards an outer layer for using in making a prosthetic socket including a planar pre-dimensioned outer layer comprises a thermoplastic material configured to be co-injected molded to make the prosthetic socket, the prosthetic socket comprising a conical cup, a lower portion and the outer layer attached to and arranged around an outer circumference of the conical cup after the co-injected molded process. The outer layer includes an inside surface, an opposite outside surface, a top, a bottom spaced apart from the top, a first side extending from the bottom to the top at a first angle measured between the bottom and the first side, and a second side opposite the first side extending from the bottom to the top at a second angle measured between the bottom and the second side, and a thickness in range from of about 0.1 mm to about 1 mm. The top includes a top region extending between the first side to the second side and the top region comprises an arch type geometry and the bottom includes a bottom region extending between the first side to the second side and the bottom region comprises an arch type geometry. The first angle is in a range from about 93 degrees to about 97 degrees and the second angle is in a range from about 93 degrees to about 97 degrees. The thermoplastic material is malleable after being heated to a shaping temperature and the outer layer and the conical cup of the prosthetic socket are configured to be substantially simultaneously formed to substantially mimic one or more contours of a residual limb or one or more contours of a model of a residual limb at the shaping temperature.

One embodiment is directed towards a reinforcement layer for a prosthetic socket including one or more layers configured to be arranged on an inner surface of at least a portion of a conical cup of the prosthetic socket. The reinforcement layer becoming stretchable after being heated at a shaping temperature and having a higher resistance against circumferential stress than the conical cup for protecting the conical cup from circumferential cracking. The reinforcement layer is configured to be shaped with the conical cup during molding of the conical cup after being heated at the shaping temperature to conform to a contour of a model.

One embodiment is directed towards a prosthetic kit including a first prosthetic socket including a first conical cup configured to enclose at least a portion of a residual limb of a patient and configured to be molded to conform to one or more contours of a model after being heated to a shaping temperature and the first conical cup having a first circumference. A second prosthetic socket including a second conical cup configured to enclose at least the portion of the residual limb of the patient and configured to be molded to conform to one or more contours of a model after being heated to a shaping temperature and the second conical cup having a second circumference different from the first circumference. The kit is also configured to allow a user to select between the first and second prosthetic sockets according to either the first circumference or the second circumference.

One embodiment is directed towards a method of forming an outer layer on a prosthetic socket. The method including obtaining a prosthetic socket, the prosthetic socket including a conical cup configured to become pliable after being heated at a shaping temperature. The conical cup is also configured to enclose at least a portion of a residual limb of a patient. The conical cup including a substantially closed first end and an open second end, the second end having an opening for facilitating placement of the portion of the residual limb into the conical cup, and a lower portion coupled to the conical cup and at an opposite end to the opening of the conical cup. Next, obtaining a substantially planar pre-dimensioned outer layer comprising a plurality of holes, an inside surface, an opposite outside surface, a top, a bottom spaced apart from the top, a first side extending from the bottom to the top at a first angle measured between the bottom and the first side, and a second side opposite the first side extending from the bottom to the top at a second angle measured between the bottom and the second side, and a thickness in range from of about 0.1 mm to about 2 mm, the top includes a top region extending between the first side to the second side and the top region comprises an arch type geometry, the bottom includes a bottom region extending between the first side to the second side and the bottom region comprises an arch type geometry, and wherein the first side is attached to the second side. The method also includes arranging the outer layer over the conical cup and heating the outer layer.

Reference will now be made in detail to an embodiment of the present invention, example of which is illustrated in the accompanying drawings.

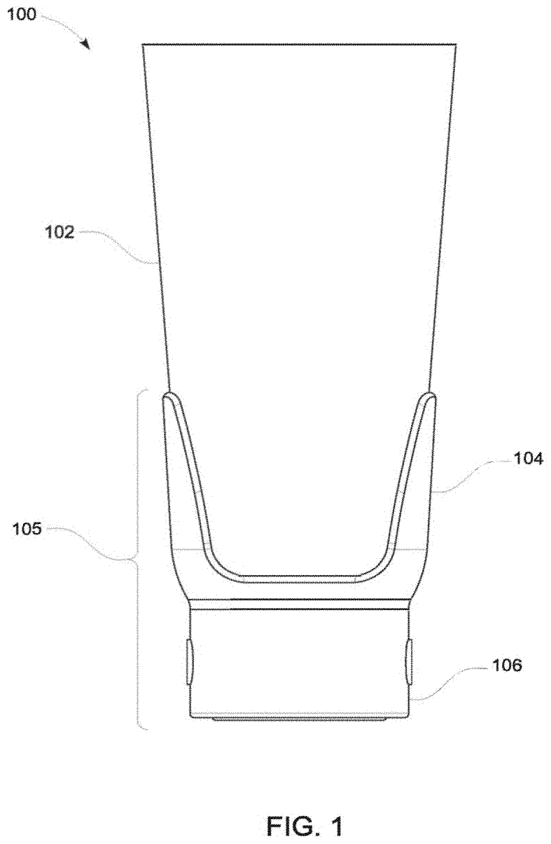

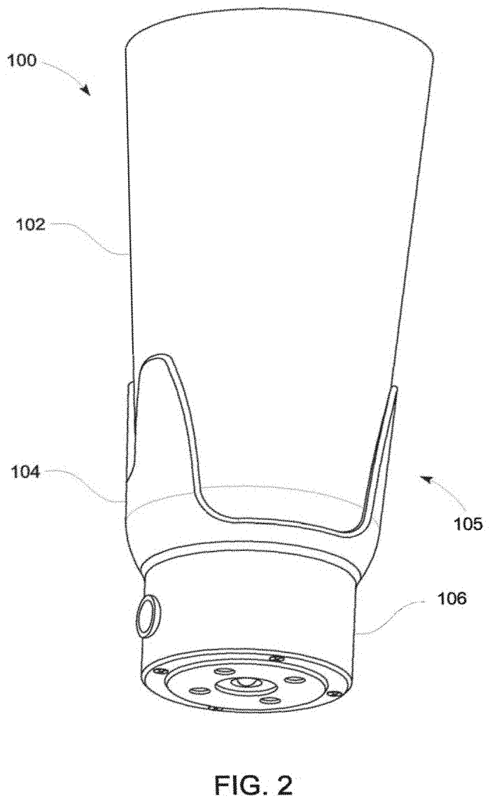

With initial reference to FIGS. 1 and 2, a prosthetic limb socket 100 in accordance with the present disclosure is illustrated. In various embodiments, the upper portion of socket 100 comprises a conical cup 102. Conical cup 102 is coupled to a lower portion 105.

Conical cup 102 is sized and configured to engage with a residual limb, securing socket 100 to the limb. Frequently, a liner is positioned around the outside of the residual limb. In such embodiments, conical cup 102 of socket 100 surrounds the liner. As noted, the liner may help reduce chafing and discomfort between the residual limb and conical cup 102, and secure them together. After conical cup 102 is positioned around and secured to the residual limb, a prosthetic limb can be attached to socket 100.

In various embodiments, conical cup 102 comprises a polymeric material as described herein. For example, conical cup 102 can be injection molded from a polymeric material. Conical cup 102 can, for example, include a polymeric material having a hardness exceeding ASTM D2240 of 70D shore hardness, a tensile strength exceeding ASTM D638 of 7,000 psi, and/or a flexural modulus exceeding ASTM D5023 of 250,000 PSI. Although described with reference to specific materials and methods of forming materials, any type of polymeric material and manner of making a suitable conical cup is within the scope of the present disclosure.

Conical cup 102 can further include, for example, a polymeric material having a pliability at a temperature in a range about 160° F. to about 305° F. and any sub-range in between, in a preferred embodiment a temperature in a range from about 225° F. to 290° F., and in more preferred embodiment a temperature in a range from about 250° F. to about 285° F. In various embodiments, when heated to between about 160° F. and about 305° F., the pliability of conical cup 102 provides a working time of between about three minutes and about 10 minutes before hardening. The pliability and working time allow conical cup 102 to be stretched circumferentially over the residual limb before conical cup 102 cools and re-hardens. Optionally and/or alternatively, the working time allows the conical cup 102 to be stretched circumferentially over a mold.

In various embodiments, conical cup 102 can include one or more additives which are added to the polymeric material to impart one or more desired physical and/or chemical properties to the polymeric material. For example, the polymeric material of conical cup 102 may comprise one or more of fiberglass, carbon fiber, aramid fiber, glass beads, carbon nanotubes, or other additives. Any additive that imparts or improves a desired physical or chemical property of the polymeric material of conical cup 102 is within the scope of the present disclosure.

Lower portion 105 includes a middle portion 104 and a base 106 positioned at the opposite end of socket 100 from conical cup 102. In various embodiments, lower portion 105 is unitary and made from a single material, such that middle portion 104 and base 106 are unitary and integral. In other embodiments, the components of lower portion 105, namely middle portion 104 and base 106, are separate and distinct from each other.

In various embodiments, middle portion 104 of lower portion 105 is coupled to conical cup 102 and base 106. Further, a prosthetic device, such as a prosthetic arm or leg, is attached and secured to base 106.

In various embodiments, a lower portion 105 (which is coupled to conical cup 102) can comprise, for example, a polymeric material. In various embodiments, the lower portion 105 includes the same polymeric material as conical cup 102. In other embodiments, lower portion 105 includes a different polymeric material than conical cup 102. For example, lower portion 105 can include a polymeric material having a second pliability that is less than the pliability of the polymeric material of conical cup 102. However, the lower portion 105 can include any suitable polymeric material.

Similar to conical cup 102, lower portion 105 can include a polymeric material having one or more additives which are added to the polymeric material to impart one or more desired physical and/or chemical properties to the polymeric material. For example, the polymeric material of lower portion 105 may include one or more of fiberglass, carbon fiber, aramid fiber, glass beads, carbon nanotubes, or other additives. Any additive that imparts or improves a desired physical or chemical property of the polymeric material of lower portion 105 is within the scope of the present disclosure.

In various embodiments, the lower portion 105 is injection molded form a polymeric material. For example, the lower portion 105 can be injection molded from the same material as conical cup 102. Further, lower portion 105 can be injection molded with conical cup 102, creating a unitary polymeric socket 100. Stated another way, polymeric socket 100 can include a one piece design where the conical cup 102 and lower portion 105 (including its components; middle portion 104 and base 106) are all made jointly and simultaneously, and are essentially a single piece).

In other embodiments, the lower portion 105 (including one or both of middle portion 104 and base 106) can be injection molded separately from conical cup 102 and secured to conical cup 102 via mechanical methods, adhesives, or any other any suitable manner of coupling the two components. In other embodiments, lower portion 105 can be over-molded, such that it is formed in contact with conical cup 102 after injection molding of conical cup 102 utilizing both parts.

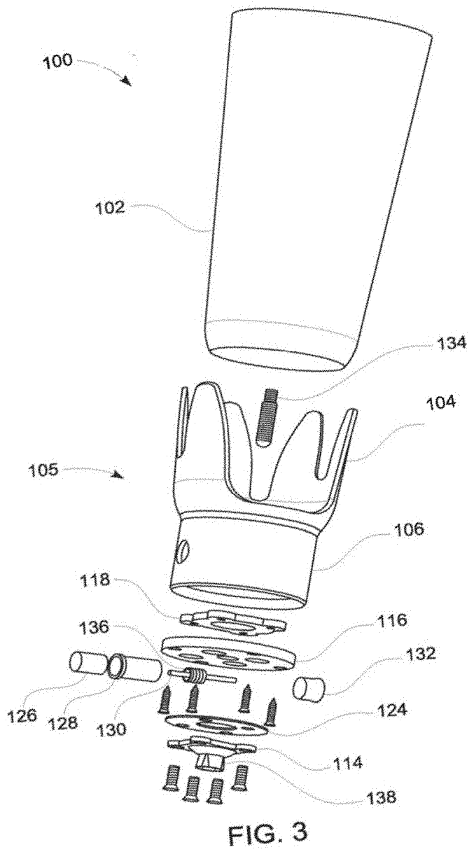

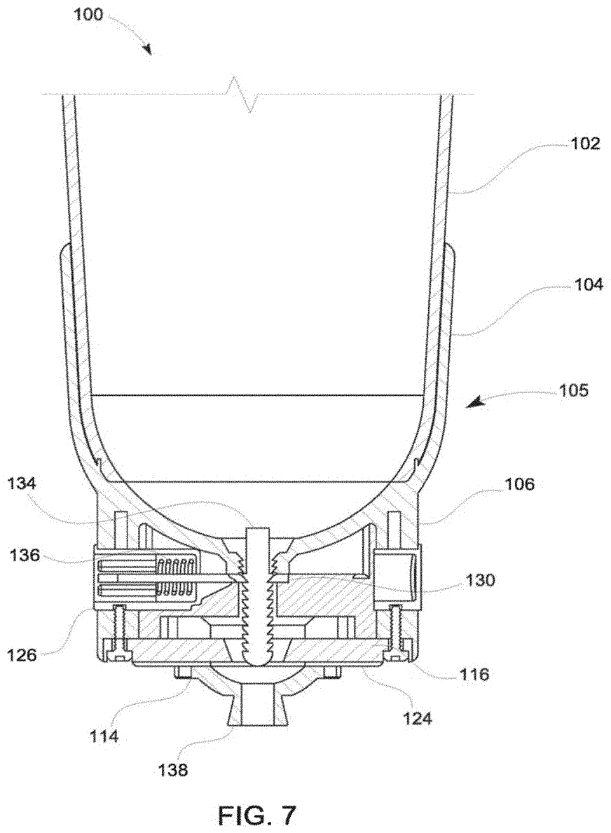

In various embodiments, base 106 is injection molded from a polymeric material, which may or may not comprise the same polymeric material from which conical cup 102 and/or lower portion 105 are formed. With initial reference to FIGS. 3, 6, and 7, socket 100 comprises means to secure a prosthetic limb to lower portion 105, and more specifically, to base 106 of socket 100. In various embodiments, socket 100 comprises a base plate 116 coupled to a locking plate 118. For example, base plate 116 can be secured to locking plate 118 by one or more threaded screws.

In various embodiments, an attachment member 114 can be secured to base plate 116. Attachment member 114 can comprise, for example, a receptacle 138 which engages with and secures a prosthetic limb to socket 100. For example, receptacle 138 can comprise one of a ball or socket, which is configured to engage with a corresponding element of a prosthetic limb to secure the limb to socket 100 in a “ball and socket” arrangement. Although described with reference to a specific physical member, any physical configuration of base plate 116 and attachment member 114 capable of coupling a prosthetic limb to socket 100 is within the scope of the present disclosure.

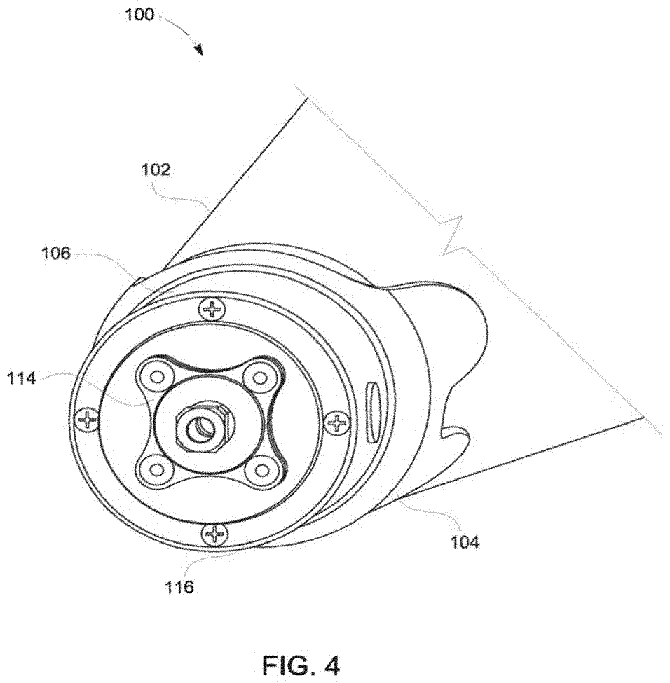

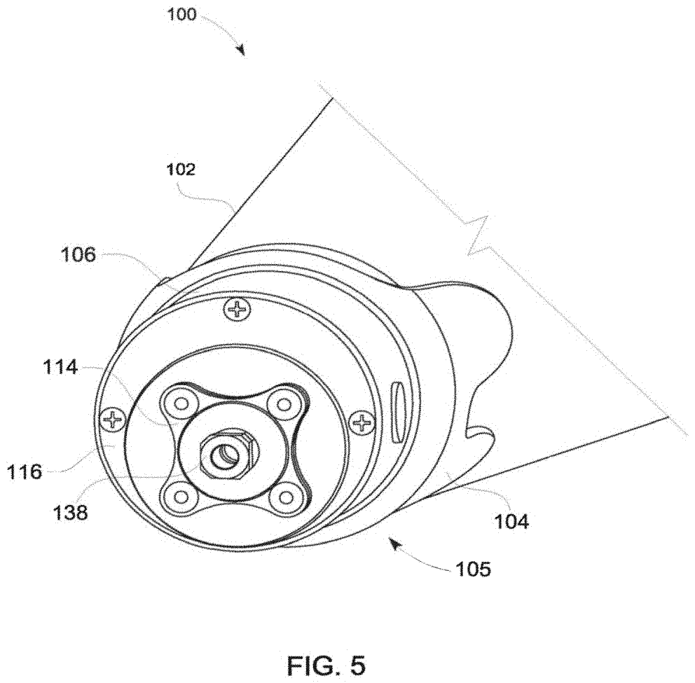

In various embodiments, locking plate 118 can slide along base plate 116. For example, by loosening the screws that secure base plate 116 to locking plate 118, locking plate 118 can slide and change orientation with regards to base plate 116, allowing attachment member 114 to change orientation relative to socket 100. With initial reference to FIG. 4, a socket 100 having a normally-oriented attachment member 114 is illustrated. In this case, normally-oriented means perpendicular to socket 100. Stated another way, base plate 116 and locking plate 118 are oriented planar to one another, such that attachment member 114 is perpendicular to socket 100. With initial reference to FIG. 5, a socket 100 having an offset attachment member 114 is illustrated. Stated another way, base plate 116 and locking plate 118 are oriented offset (or non-planer) to one another, such that attachment member 114 is not perpendicular to (i.e., is offset from) socket 100. Such adjustment allows for the prosthetic limb coupled to attachment member 114 to be oriented properly relative to the residual limb of the patient.

Socket 100 can further comprise, for example, a cover 124 positioned proximal base 106. For example, cover 124 can be positioned at or near the bottom of base 106 and can prevent dirt or other contaminants from entering base 106 and socket 100.

In various embodiments, socket 100 can further comprise a typical locking pin 134 that couples the liner to socket 100. In various embodiments, locking pin 134 is secured to socket 100 by a pin plate 130. For example, pin plate 130 can move laterally with regards to locking pin 134 by, for example, button 126, which engages and disengages pin plate 130 from locking pin 134. Locking pin 134 may, for example, comprise ridges that engage with pin plate 130, such that as locking pin 134 of the liner is inserted into the locking mechanism of base 106, it click locks incrementally, securing the liner into base 106 and socket 100. In various embodiments, button 126 is housed within button housing 128. Further, a housing cover 132 may be positioned on a side of button housing 128 opposite button 126. Pin plate 130 can further comprise a spring 136.

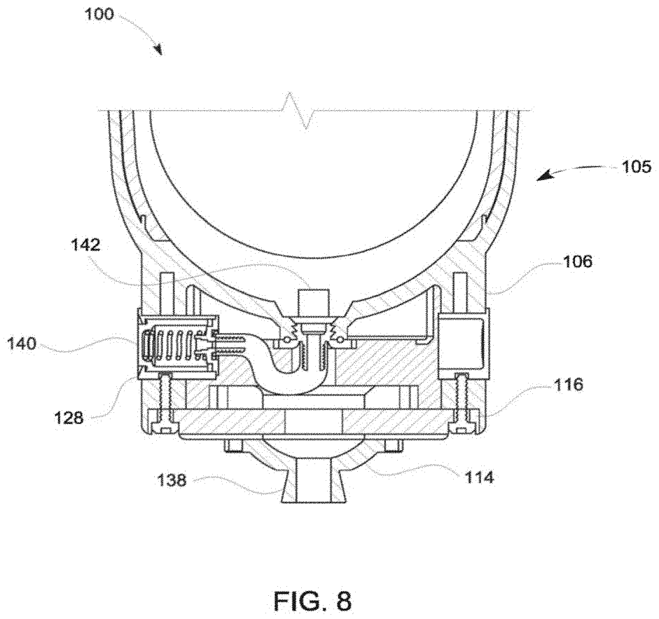

With initial reference to FIG. 8, another embodiment of socket 100 is illustrated. For example, socket 100 can comprise a vacuum port 140 configured to apply vacuum through vacuum outlet 142 against a gel liner attached to a patient. Vacuum port 140 can be positioned in housing 128 (previously referred to as button housing 128 in connection with other embodiments), and can be quickly and easily accessed by the patient and prosthetists to apply or reduce suction to socket 100, allowing for securement or removal of socket 100 from the liner. Although described with reference to specific physical configurations, any manner of coupling socket 100 to a prosthetic liner is within the scope of the present disclosure.

Numerous methods of securing socket 100 to a liner (and thus the residual limb) can be utilized. For example, the following methods are also within the scope of the present disclosure; The liner has a soft ring shaped ridge near the bottom (not shown) that creates an air tight seal to the interior of socket 100 and conical cup 102. As the residual limb is pushed into conical cup 102, the air is expelled through a valve at base 106. The liner is tightly held to socket 100 by suction. To remove socket 100 form the residual limb, a button is depressed allowing air into socket 100 and breaking the suction.

A sleeve (not shown) is placed over the top of conical cup 102 of socket 100, extending upwards over at least a portion of the residual limb. The sleeve fits tight to form an air tight seal between socket 100 and the skin of the residual limb. This creates suction holding the liner to socket 100. The sleeve is rolled down the residual limb and socket 100 to break the seal, allowing socket 100 to be removed; and

A sleeve can be used to seal socket 100 to the residual limb in conjunction with a vacuum pump that creates negative pressure, sucking the liner into conical cup 102 of socket 100. These pumps can be hand activated, activated by walking, or by using an electro-mechanical pump to create suction.

Socket 100 can further comprise, for example, a coupling member to couple and secure socket 100 to a liner of a residual limb. In various embodiments, the coupling member attaches base 106 to the liner. Such coupling members can comprise, for example, a clip (such as a spring clip) that engages a locking post of the gel liner, an air tight button seal to allow air to escape, be sealed, and be released for use with a suction socket retention system, and/or a hose attachment that couples to a vacuum source that applies negative pressure between base 106 and the gel liner. Although described with reference to specific mechanical member, any manner of coupling socket 100 with a prosthetic limb is within the scope of the present disclosure.

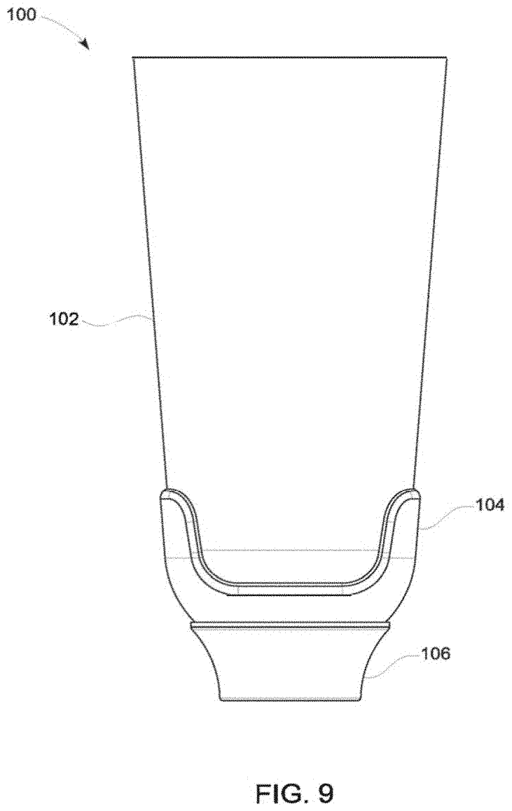

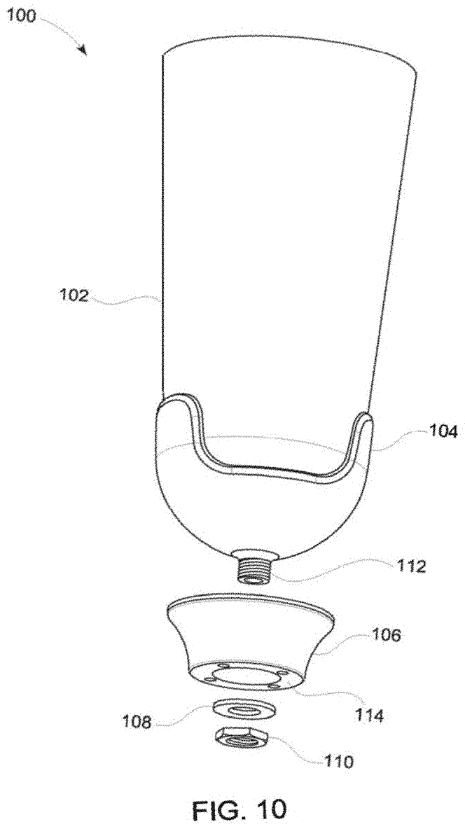

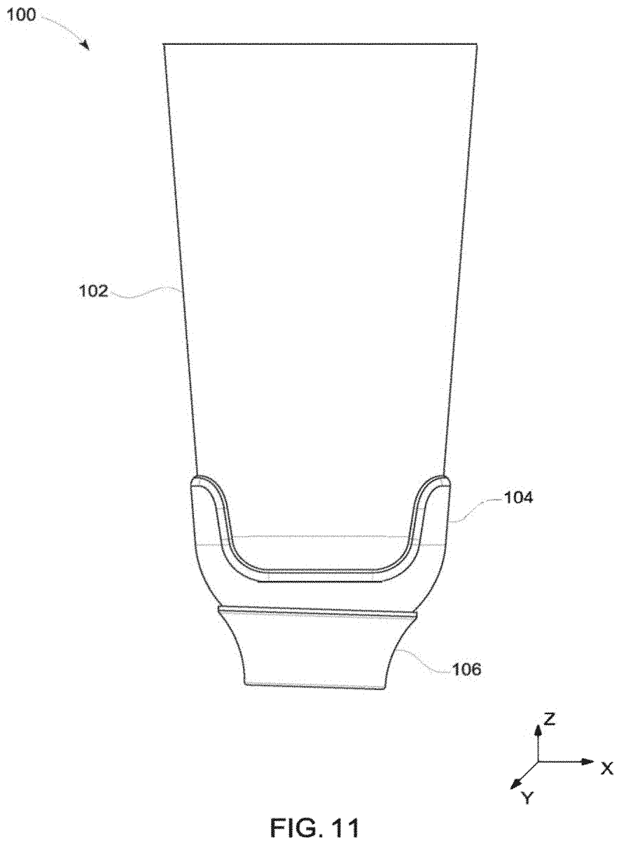

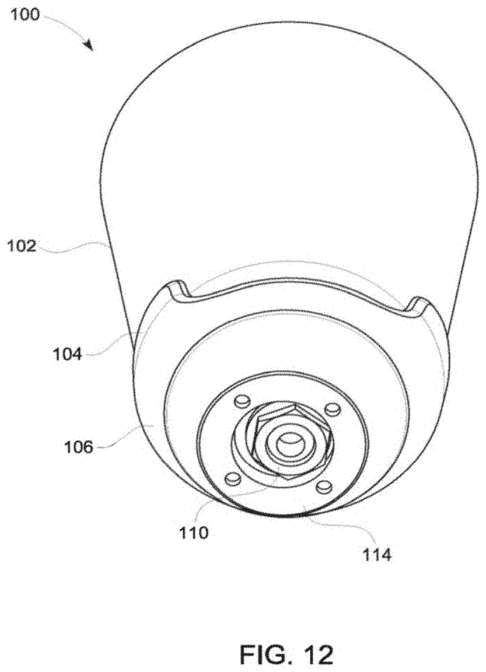

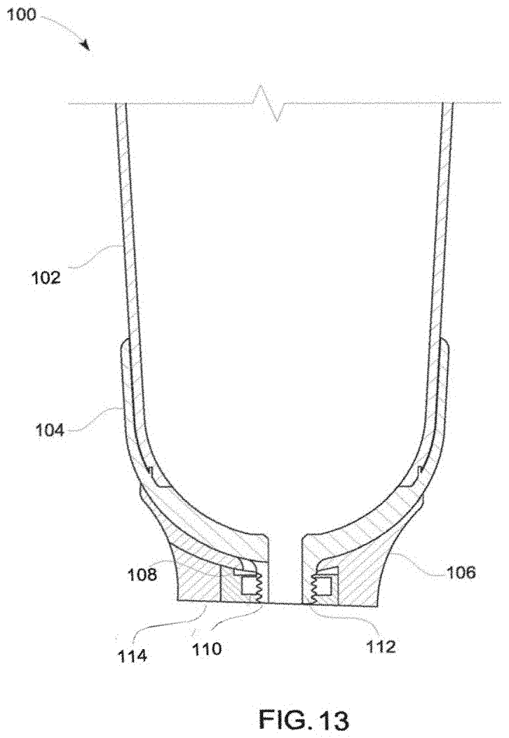

With initial reference to FIGS. 9-13, in various embodiments, base 106 is secured to middle portion 104 via a fastening member 112. For example, a fastening member 112 can be formed into or otherwise secured to a bottom surface of middle portion 104 or conical cup 102. In various embodiments, fastening member 112 passes through a portion of base 106 and is secured by a corresponding member 110, holding base 106 in position relative to middle portion 104. For example, fastening member 112 can comprise a threaded portion protruding from middle portion 104 into base 106. A washer 108 and nut 110 are coupled to fastening member 112, which secures base 106 to middle portion 104. Although described with reference to specific arrangements, any manner of coupling base 106 to middle portion 104 or conical cup 102 is within the scope of the present disclosure.

In various embodiments, an attachment member 114 can be secured to base 106 by fastening member 112 and corresponding member 110. For example, attachment member 114 can be positioned along fastening member 112 (e.g., a threaded portion) and secured via member 110 (e.g., a nut). Although described with reference to the various drawing figures and specific embodiments, any manner of securing attachment member 114 to socket 100 is with the scope of the present disclosure.

For example, base 106 can comprise a mechanical attachment member 114 to couple a component of the prosthetic arm or leg to socket 100. In various embodiments, attachment member 114 comprises a baseplate which attaches to the prosthetic limb by, for example, screws.

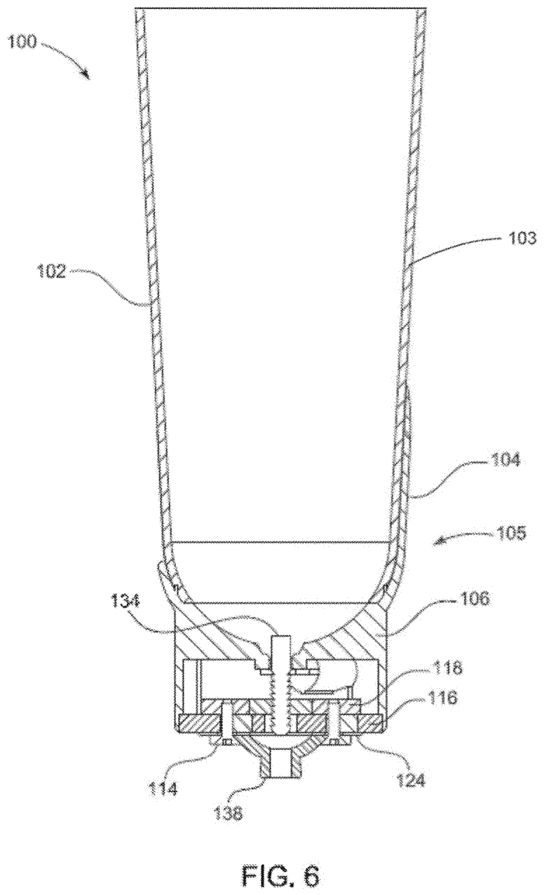

In various embodiments, attachment member 114 is adjustable in one or more planes or directions. With reference to FIGS. 6-8, attachment member 114 of base 106 allows for positional adjustment of socket 100 and the prosthetic limb with respect to each other. For example, the prosthetic limb may be offset horizontally from a center axis of socket 100 in order to properly orient and position the socket 100 and the prosthetic limb for optimal use, gait, and/or balance. In various embodiments, attachment member 114 comprises a baseplate which attaches to the prosthetic limb in a manner that allows for angular adjustment between base 106 and the prosthetic limb. For example, the adjustment member 114 may be bolted to the prosthetic limb, and loosening the bolts can allow for the base plate to be slid and adjusted in a planar horizontal manner to offset the prosthetic limb as desired. Tightening the bolts locks base 106 and the prosthetic limb in position relative to one another.

In various embodiments, socket 100 further comprises an insert layer. In such embodiments, the insert layer can be positioned within conical cup 102, and comprises a material that causes conical cup 102 to draw tight circumferentially over the residual limb as it is heated and formed around the limb. In various embodiments, an insert layer can comprise one or more of a knit fabric, a mesh, and/or a thin sheet or perforated material of stretch rubber, polyurethane, estane, spandex, or long chain polymer. Although described with reference to specific materials, an insert layer can comprise any material suitable for circumferentially tightening a portion of socket 100 (such as conical cup 102) when heated.

Further, an insert layer can comprise a protruding portion that extends upward from the opening of conical cup 102. In such embodiments, for example, the protruding portion may comprise one or more handles, which can be used to help pull socket 100 onto the residual limb during forming.

In various embodiments, socket 100 can further comprise an outer layer that surrounds at least a portion of socket 100. The outer layer may provide a durable, smooth outer surface of socket 100, and may be printable and/or textured for cosmetic purposes. For example, the outer layer can be co-molded or adhered to an outer surface of socket 100 (such as, for example, an outer surface of conical cup 102). In various embodiments, the outer layer comprises a sheet material, such as a PET material (polyethylene terephthalate), a polyester material, a vinyl material, a PVC material or other plastic that becomes pliable and stretchable at a temperature in a range from about 160° F. to about 305° F. and any sub-range in between, in a preferred embodiment a temperature in a range from about 225° F. to 290° F., and in more preferred embodiment a temperature in a range from about 250° F. to about 285° F. Further, the outer layer can have a thickness, for example, between about 0.005 inches and about 0.02 inches (about 0.12 mm and about 0.31 mm). The outer layer can also be a clear polymer, e.g., optically clear. The outer layer becomes slightly less formable than the body of the socket 100 at the forming or shaping temperatures so as to create a skin that aids in smoothly forming the outer surface. This surface resists finger or glove prints when forming and produces a very smooth and appealing surface. It can also have a gloss finish that is more durable and scratch resistant than the socket 100.

In various embodiments, methods of forming sockets 100 to residual limbs include selecting the appropriate socket size. For example, conical cup 102 of socket 100 can include a circumference that is smaller than the circumference of the residual limb. In such embodiments, when socket 100 and conical cup 102 are heated, the material of socket 100 becomes sufficiently pliable and stretchable to allow conical cup 102 to be stretched over the residual limb or model. As socket 100 cools, conical cup 102 contracts to its pre-heated circumference, providing a circumferentially tight fit to the residual limb.

Socket 100 and conical cup 102 can be sized using a set of pre-sized plastic or foam cups that are used to measure the residual limb having a liner in place. For example, the different sized cups can include a label with the corresponding suggested socket size that is smaller in circumference than the sizing cup, so as to achieve the correct percentage of reduction in circumference of socket 100 and conical cup 102 after heating, resulting in a proper tight fit.

In various embodiments, spaces or voids can be created within socket 100 to correspond with sensitive portions of the residual limb. For example, padding such as foam pieces, tapered gel pads, cotton wadding or other forms can be applied directly to the skin and placed under the gel liner worn by the amputee, creating extra space within socket 100 during the heat forming process. The padding can be removed after cooling and hardening of socket 100.

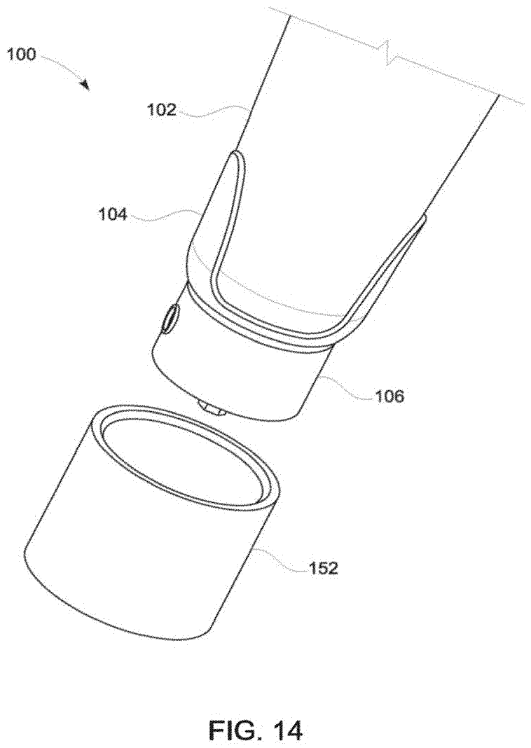

Sockets 100 in accordance with the present disclosure are fitted to residual limbs by heating a portion of socket 100 to a predetermined temperature, allowing the portion of socket 100 to be plastically deformed to conform to the residual limb. In various embodiments, socket 100 is differentially heated so that conical cup 102 is heated to a temperature in a range from about 225° F. to about 275° F. (107° C. to 135° C.), while base 106 remains at or near room temperature. For example, with reference to FIG. 14, an insulating cup 152 may be fitted to a portion of lower section 105 (for example, base 106) to reduce heat transferred into the portion of lower section 105 and preventing any change in shape of the portion. Suitable insulating cups 152 can comprise, for example, molded foam, fabric insulative batting, silicone, or any material capable of adequately insulating a portion of lower section 105, and capable of withstanding repeated heating cycles without deteriorating. After sufficiently heating socket 100, insulating cup 152 may be removed from socket 100.

In various embodiments, a method for applying socket 100 can further comprise applying an insulating cover (not shown) over the outer surface of a portion of socket 100. For example, an insulating cover can be applied before or after heating, and can apply circumferential compression to the heated section of socket 100 (such as, for example, conical cup 102). The insulating cover may prevent heat loss from socket 100, extending the working time of socket 100 and allowing for more time to fit socket 100 to a residual limb.

For example, the insulating cover may comprise a tubular or cup shaped cover comprising a stretch insulating material such as neoprene foam with a stretch fabric covering such as wetsuit material, closed-cell foam, knit stretch fabric, spandex fabric and the like. Further, the insulating cover can comprise strapping applied vertically and extending above the top of the cover to provide handles for pulling the socket onto the residual limb. Once socket 100 is properly installed on the residual limb, the insulating cover can be removed from socket 100.

Methods for applying socket 100 can further include applying an outer sleeve around a portion of socket 100. For example, a woven or knit outer sleeve can be fitted around a portion of socket 100 before or after heating. In various embodiments, the outer sleeve can apply pressure to socket 100 such that the circumference of a portion of socket 100 (such as, for example, conical cup 102) is reduced as the outer sleeve is stretched vertically. In such embodiments, the outer sleeve operates similarly to a Chinese finger trap. The outer sleeve can comprise a material woven in a diagonal pattern.

Further, the outer sleeve can be suspended above socket 100 by a framework. For example, the framework can comprise a hoop from which the outer sleeve is suspended, in a configuration similar to a basketball hoop and net. The opposite (e.g., bottom) end of the outer sleeve can comprise a rigid ring that fits over the base 106, thereby locking it to outer sleeve. In such embodiments, after heating socket 100 to the working temperature, the residual limb is inserted into socket 100, and the downward pressure applied by the residual limb vertically extends the outer sleeve, causing it to circumferentially compress pliable socket 100 around the residual limb.

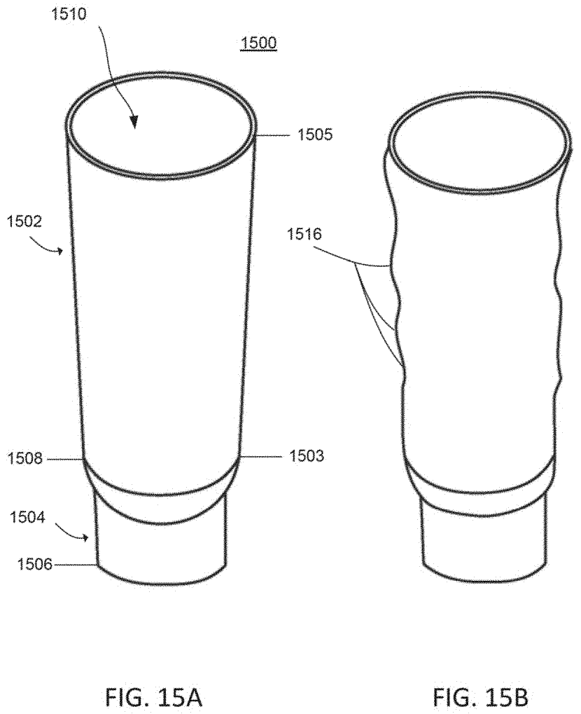

FIG. 15A is a perspective view of a prosthetic socket in accordance with an embodiment. FIG. 15B is a perspective view of a prosthetic socket of FIG. 15A after being heated in accordance with an embodiment.

Referring to FIGS. 15A-15B, the prosthetic socket is generally represented with reference to number 1500. The prosthetic socket 1500 is used to join a residual limb to a prosthesis. For example, a lower calf portion of a residual limb can sit inside the prosthetic socket 1500 that attaches to a prosthetic mechanical lower leg to allow a person with a below or above the knee amputation to walk using the socket plus prosthesis. The prosthetic socket 1500 includes a conical cup 1502 and a lower portion 1504. In other embodiments, the prosthetic socket 1500 can include different components. The conical cup 1502 is in the shape of a hollow deep or elongated cup that is generally cylindrical in shape having a first end 1503 and second end 1505. The lower portion 1504 includes a first end 1506 and a second end 1508. The lower potion is configured also includes a cup region to receive a portion surrounding a portion of the conical cup 1502.

The conical cup 1502 is in the shape of a hollow deep or elongated cup that is generally cylindrical in shape having a first end 1503 and second end 1505. The conical cup 1502 is formed with an opening 1510 via which the residual limb can be inserted into the conical cup 1502. The conical cup 1502 is shapeable or moldable after being heated at a shaping temperature. The shaping temperature can be in the range from about 120° F. to about 300° F. and any sub-range within. In some embodiments, the conical cup 1502 has a pliability above a threshold pliability for a shaping time after being heated at the shaping temperature. The shaping time can be in the range of about 5 minutes to about 15 minutes, or any sub-range within. In one embodiment, during the shaping time, the conical cup 1502 can be stretched in various dimensions, e.g., circumferentially, over the residual limb or a model of the residual limb so that the conical cup 1510 is shaped to fit the residual limb.

In this embodiment, the conical cup 1502 does not include one or more outer layers. After heating to the shaping temperature, it forms a wavy, bumping, uneven surface 1516 and become deformed as shown in FIG. 15B. It has been found this a wavy, bumping, uneven surface 1516 is present upon cooling as well. The surface also shows handling marks, e.g., finger and hand marks.

In some embodiments, the lower portion can be formed with blow molding, injection molding, rotational molding or other techniques. In one embodiment, the lower portion 1504 is formed with a material that is not moldable at a shaping temperature. The lower portion 1504 includes a material more rigid than the conical cup portion 1504 at a shaping temperature or room temperature. The lower portion includes a material such as, acrylonitrile butadiene styrene (ABS), nylon, polycarbonate, or a fiberglass or carbon filled polymer.

Optionally and/or alternatively, the lower portion 1504 is made from the same material as the conical cup 1502. In one embodiment, the lower portion 1504 is not heated or is heated at a lower temperature than the shaping temperature when the conical cup 1502 is heated, such as a temperature below about 250° F. The lower portion 1504 and the conical cup can be one piece.

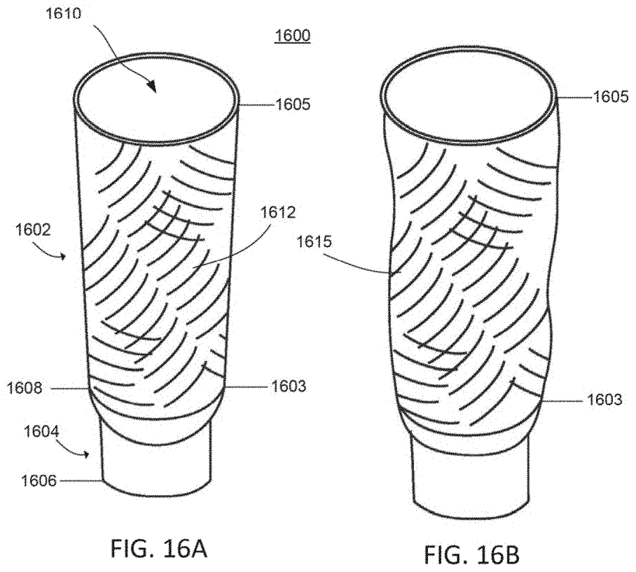

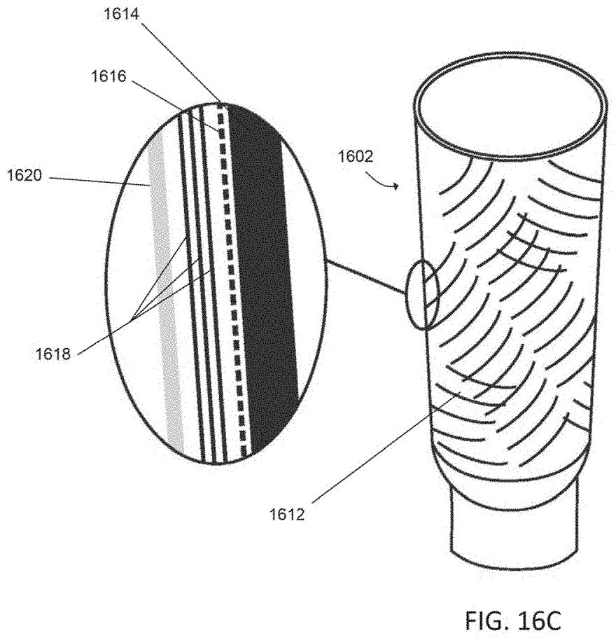

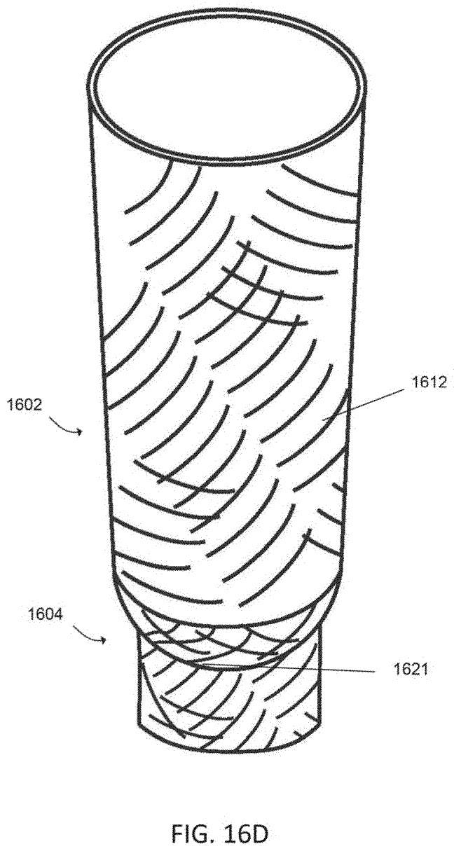

FIG. 16A is a perspective view of a prosthetic socket in accordance with an embodiment. FIG. 16B is a perspective view of a prosthetic socket of FIG. 16A after being heated in accordance with an embodiment. FIG. 16C is a cross-sectional view of FIG. 16A. FIG. 16D is a perspective view of a prosthetic socket in accordance with an embodiment with an outer layer.

Referring to FIGS. 16A-16B, the prosthetic socket is generally represented with reference to number 1600. The prosthetic socket 1600 is used to join a residual limb to a prosthesis. For example, a lower calf portion of a residual limb can sit inside the prosthetic socket 1600 that attaches to a prosthetic mechanical lower leg to allow a person with a below or above the knee amputation to walk using the socket plus prosthesis. The prosthetic socket 1600 includes a conical cup 1602 and a lower portion 1604. In other embodiments, the prosthetic socket 1600 can include different components.

The conical cup 1602 is in the shape of a hollow deep or elongated cup that is generally cylindrical in shape having a first end 1603 and second end 1605. It has an outer surface and an inner surface. The conical cup 1602 is formed with an opening 1610 via which the residual limb can be inserted into the conical cup 1602. The conical cup 1602 is shapeable or moldable after being heated at a shaping temperature. The shaping temperature can be in a range of about 160° F. to about 305° F. and any sub-range within. In some embodiments, the conical cup 1602 has a pliability above a threshold pliability for a shaping time after being heated at the shaping temperature. In a preferred embodiment, the shaping time can be in the range of about 2 minutes to about 10 minutes, or greater and any sub-range within.

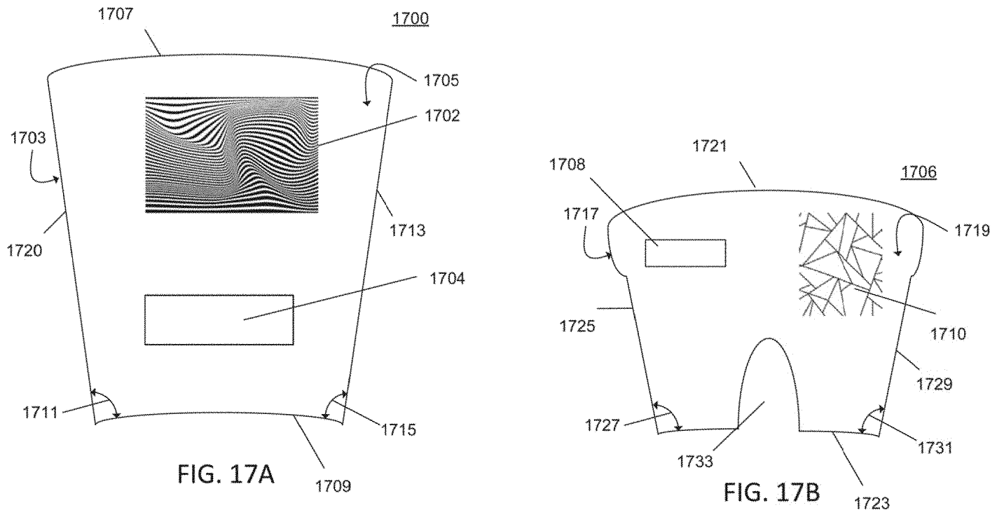

In this embodiment, the conical cup 1602 includes one or more outer layers 1612. The outer layer 1612 is described in more detail with regard to FIG. 17A and FIG. 17B. The outer layer may include any graphical design, graphical pattern, color, logo, and combinations of the same. In one embodiment the design is applied with ink applied by printing, screen printing, digital printing or other method as known in the art. A heat activated adhesive material can then applied over the design. The graphical layer or layers printed onto the side of a clear outer layer that faces the socket 100 shows through the clear outer layer, and then the outer layer protects the graphic ink from being scratched or abraded. The outer layer and optionally inks may include ultraviolet (UV) protectant to aid with discoloration from the UV rays from the sun

Referring to FIG. 16C, the conical cup 1602 has a sidewall 1614 with a thickness in a range from about 1 mm to about 5 mm or greater and any range in between. The outer layer 1612 may include any graphical design, graphical pattern, color, logo, and combinations of the same that is printed by a liquid ink or inks 1618 applied by printing, screen printing, digital printing or other method that dries. The inks 1618 are printed onto the side of the clear outer layer that faces the socket 100 shows through the clear outer layer, and then the outer layer protects the graphic ink from being scratched or abraded. The inks 1618 can be any ink known in the art, e.g., metallic or reflective ink, thermochromic ink that changes color with changes in temperature. A heat activated adhesive 1616 is then applied over the ink layers. The adhesive layer 1616 includes, e.g., a liquid applied by printing, screen printing, digital printing or other method that dries and becomes a thermoset adhesive activated by heat at the forming process. The protective layer 1620 can be clear, shaded, or have any sheen, e.g., matte, glossy, and semi-gloss. The layer 1612 can be made from a thermoplastic material or polymer, e.g., polyethylene terephthalate (PET), Polyethylene, Polypropylene (PP), Polystyrene or Styrofoam (PS), combinations of the same and the like.

In one embodiment, during the shaping time, the conical cup 1602 can be stretched in various dimensions, e.g., circumferentially, over the residual limb or a model of the residual limb so that the conical cup 1602 is shaped to fit the residual limb.

The outer layer 1612 is arranged on the outer surface of the conical cup and adheres to the outer surface of the conical cup during the fabrication process, e.g., Injection molding, rotational molding, blow molding. The outer layer is malleable after being heated to a shaping temperature as shown in FIG. 16B. The outer layer comprises one or layer 1612 and materials as described herein. When the outer layer 1612 is heated to a shaping temperature, the conical cup is contained within the outer layer and able to be molded to contours of a residual limb or a model of a residual limb but non-malleable upon cooling down from the shaping temperature. It has a higher rigidity than the conical cup 1602 and can protect the conical cup 1602 from damages, such as cracks that occur during molding of the prosthetic socket 1600. The outer layer 1612 is fingerprint resistant at the shaping temperature, so that during hand molding of the prosthetic socket, no fingerprint is left on the outer layer 1612.

The thickness of the outer layer 1612 can be in a range from 0.01 mm to 2 mm or greater. In a preferred embodiment, the outer layer 1612 has a thickness in a range from about 0.1 to 1.5 mm or greater. The outer layer 1612 can be less malleable than the conical cup at the shaping temperature. Comparing the conical cup 1502 of FIG. 15B and conical cup 1602 of FIG. 16B, after heating, the conical cup 1602 has less wavy or uneven patterns and becomes less deformed to the conical cup 1502 in FIG. 16B.

The outer layer 1612 has a higher rigidity than the conical cup to protect the conical cup from damages, such as damages could occur during producing the prosthetic socket. Also, the outer layer 1612 has smoother outer surface than the outer surface of the conical cup. The outer layer 1612 can be scratch resistant at room temperature. In some embodiments, the outer layer and the conical cup are co-injection molded. Moreover, referring to FIG. 16B after heating and forming the surface 1615 is smooth and evenly formed without marring or handling marks. The outer surface is robust and abrasion resistant.

Optionally and/or alternatively, a reinforcement layer (not shown) can also be arranged on the inner surface or outer surface of the conical cup during a molding process. It has a higher resistance against circumferential stress than the conical cup. The reinforcement layer can have a woven fiber structure. It includes a fiber material, such as carbon fiber, kevlar, glass fiber, or some combination thereof.

The lower portion 1604 joins the conical cup 1602 to the prosthesis. The lower portion or base 1604 can have a pliability that is a lower than the pliability of the conical cup 1602 at the shaping temperature and/or room temperature. Optionally and/or alternatively, the lower portion 1604 is made from the same material as the conical cup 1602. The lower portion 1604 is not heated or is heated at a lower temperature than the shaping temperature when the conical cup 1602 is heated, such as a temperature below about 250° F. The lower portion 1604 and the conical cup can be one piece. The lower portion 1604 includes a first end 1606 and a second end 1608. In some other embodiments, the lower portion 1604 is made from a different material than the conical cup 1602. Referring to FIG. 16D, optionally and/or alternatively, the lower portion 1604 includes an outer layer 1621. The outer layer 1621 is described herein with reference to FIG. 17B.

FIG. 17A is top view of an outer layer configured to be attached to a conical cup of a prosthetic socket according to an embodiment. FIG. 17B is top view of an outer layer configured to be attached to a lower portion prosthetic socket according to an embodiment.

Referring to FIG. 17A, the outer layer 1700 is cut into a shape and dimension configured to precisely fit to the outer portion of the conical cup 1602 that can be insert molded or applied to the outside of the conical cup 1602. Referring to FIG. 17B, an outer layer 1706 has a predetermined, pre-dimensioned or pre-cut geometry that is then formed to a die that has the same shape as the lower portion of the prosthetic socket by heating and vacuum forming to substantially mimic the three dimensional shape of the lower portion of the conical cup 1602 including the circumference. In one embodiment, when making the lower portion of the prosthetic socket while injection molding, the outer layer shown in 17B can be configured into the injection mold. Next, the injection mold is operated and the outer layer 1700 is molded into integral unit, e.g., as shown in FIG. 16A.

The outer layer 1700 has a pre-dimensioned geometry to substantially mimic the dimensions of the conical cup 1602 including the circumference. In one embodiment, when making the conical cup with injection molding the outer layer 1700 can be configured into the injection mold. Next, the injection mold is operated and the outer layer 1700 is molded into integral unit 1600, e.g., as shown in FIG. 16A.

The outer layer 1700 includes an inside surface 1703, an opposite outside surface 1705, a top 1707, a bottom 1709 spaced apart from the top 1707, a first side 1720 extending from the bottom 1709 to the top 1707 at a first angle 1711 measured between the bottom 1709 and the first side 1720, and a second side 1713 opposite the first side 1720 extending from the bottom 1709 to the top 1707 at a second angle 1715 measured between the bottom 1709 and the second side 1713. The outer layer 1700 also has a thickness in a range from of about 0.1 mm to about 1 mm.

The first angle 1711 can be in a range from about 91 degrees to about 120 degrees, in a preferred embodiment the first angle 1711 is in a range from about 95 degrees to about 105 degrees. The second angle 1715 can be in a range from about 91 degrees to about 120 degrees, in a preferred embodiment the second angle 1715 is in a range from about 95 degrees to about 105 degrees. Referring to FIG. 17B, the outer layer 1706 is used for a lower portion 1604 of the prosthetic socket 1600 in accordance with an embodiment. The outer layer 1706 has a pre-dimensioned geometry to substantially mimic the dimensions of the lower portion 1604. In one embodiment, when making the conical cup 1602 with injection molding the outer layer 1700 can be configured into the injection mold. Next, the injection mold is operated and the outer layer 1700 is molded into an integral unit, e.g., as shown in FIG. 16A.

As described herein the outer layer may include more than one outer layer 1700. The outer layer 1700 first surface includes an adhesive layer. In a preferred embodiment, the outer layer 1700 includes any graphical design, graphical pattern, color, logo, and combinations of the same 1702. In one embodiment, the outer layer 1700 also includes a logo, branding, trademark and combinations of the same 1704.

Referring not to FIG. 17B, the outer layer 1706 is used for a lower portion 1604 of the prosthetic socket 1600 in accordance with an embodiment. The outer layer 1706 has a pre-dimensioned geometry to substantially mimic the dimensions of the lower portion 1604 including the circumference. In one embodiment, when making the prosthetic socket 1600 with injection molding the outer layer 1706 can be configured into the injection mold. Next, the injection mold is operated and the outer layer 1706 is molded into integral unit of the lower portion 1604 as an outer layer 1706 on the lower portion. The outer layer can have a thickness of 0.5 mm to about 3 mm or greater.

The outer layer 1706 includes an inside surface 1717, an opposite outside surface 1719, a top 1721, a bottom 1723 spaced apart from the top 1721, a first side 1725 extending from the bottom 1723 to the top 1721 at a first angle 1727 measured between the bottom 1723 and the first side 1725, and a second side 1729 opposite the first side 1725 extending from the bottom 1723 to the top 1721 at a second angle 1731 measured between the bottom 1723 and the second side 1729. The outer layer 1706 also has a thickness in a range from of about 0.1 mm to about 1 mm. The bottom has a cutout 1733 or void to mimic a cutout or void on the lower portion (not shown).

The first angle 1727 can be in a range from about 91 degrees to about 120 degrees, in a preferred embodiment the first angle 1727 is in a range from about 95 degrees to about 105 degrees. The second angle 1731 can be in a range from about 91 degrees to about 120 degrees, in a preferred embodiment the second angle 1731 is in a range from about 95 degrees to about 105 degrees.

As described herein the 1706 outer layer may include more than one outer layer 1706. The outer layer 1706 has a first surface that includes one or more of an adhesive layer, adhesive pattern, and combinations of the same. In a preferred embodiment, the outer layer 1706 includes any graphical design, graphical pattern, color, logo, and combinations of the same 1710. In one embodiment, the outer layer 1708 also includes a logo, branding, trademark, and combinations of the same 1710.

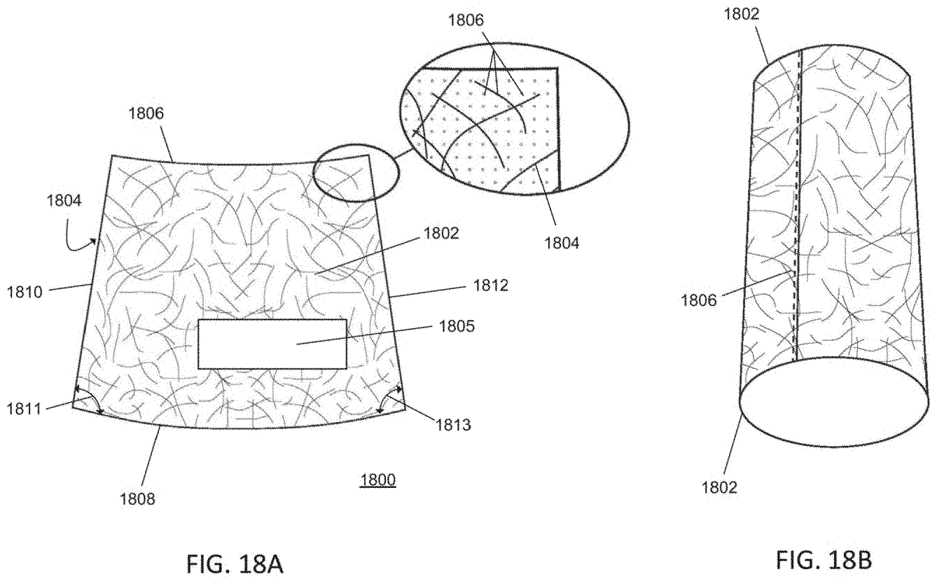

FIG. 18A is a top view of an outer layer configured to be attached to a conical cup of a prosthetic socket according to an embodiment. FIG. 18B is a side view of the outer layer of FIG. 18A in a second orientation.

Referring to FIGS. 18A-18B, an outer layer 1800 is formed, e.g., cut, laser cut, into a shape and dimension configured to precisely fit to the outer portion of the conical cup 1902 portion of the prosthetic socket. The pre-dimensioned outer layer 1800 has a geometry to substantially mimic the dimensions of the conical cup 1904 including the circumference, height, diameter, etc.

The outer layer 1800 includes a first surface 1802, an inside surface 1804, a top 1802, a bottom 1806 spaced apart from the top 1808, a first side 1810 extending from the top 1808 to the bottom 1806 at a first angle 1811 measured between the bottom 1808 and the first side 1810, and a second side 1812 opposite the first side 1810 extending from the top 1808 to the bottom 1806 at a second angle 1813 measured between the top 1808 and the second side 1812. The outer layer 1800 also has a thickness in a range from of about 0.1 mm to about 1 mm or greater.

The first angle 1811 can be in a range from about 91 degrees to about 120 degrees, in a preferred embodiment the first angle 1811 is in a range from about 93 degrees to about 105 degrees. The second angle 1813 can be in a range from about 89 degrees to about 60 degrees, in a preferred embodiment the second angle 1813 is in a range from about 85 degrees to about 70 degrees.

As described herein the outer layer 1800 may include more than one outer layer. The outer layer 1800 surface 1804 includes an adhesive layer. In a preferred embodiment, the outer layer 1800 includes any graphical design, graphical pattern, color, logo, and combinations of the same 1802. In one embodiment, the outer layer 1800 also includes a logo, branding, trademark and combinations of the same 1805. Optionally and/or alternatively the logo 1805 can include or include an addition radio-frequency identification (RFID) tag, e.g., active or passive as known in the art. The RFID tag may contain information about the product, e.g., manufacturer, manufacturing date, and other information.

In one embodiment, the outer layer 1800 is described with reference to FIG. 16C and can include any graphical design, graphical pattern, color, logo, and combinations of the same that is printed by a liquid ink or inks 1618 applied by printing, screen printing, digital printing or other method that dries. The inks 1618 can be any ink known in the art, e.g., metallic or reflective ink, thermochromic ink that changes color with changes in temperature. A heat activated adhesive 1616 over the ink layers 1618. The adhesive layer 1616 includes, e.g., a liquid applied by printing, screen printing, digital printing or other method that dries and becomes a thermoset adhesive activated by heat at the forming process. A protective layer 1620 can be clear, shaded, or have any sheen, e.g., matte, glossy, and semi-gloss. The outer layer 1800 can be made from a thermoplastic material or polymer, e.g., polyethylene terephthalate (PET), polyethylene, polypropylene (PP), polystyrene or styrofoam (PS), combinations of the same and the like.

The outer layer 1800 includes a plurality of holes 1806. The holes go completely through the outer layer 1800. The holes 1806 can be formed with a laser in any pattern, e.g., linear, non-linear, grid, etc. The holes have a dimension of about 0.004 inches and can be in a range from about 0.001 inches to about 0.009 inches or larger. The holes can be characterized as micro perforations. The can be made by laser after the outer layer has the required graphics. The first side 1810 of the outer layer 1800 is attached to the second side 1812 as shown in FIG. 18B. The attachment can include a partial overlap of the second side 1812 over the first side 1810 or no overlap. The attachment can be any one of an adhesive, a laser bond, weld, e.g., ultrasonically weld, heat weld, other attachment technique and combinations of the same.

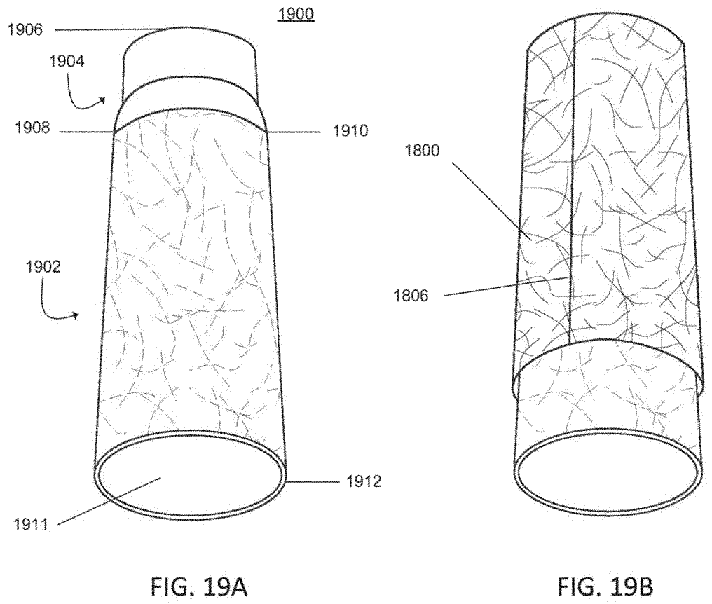

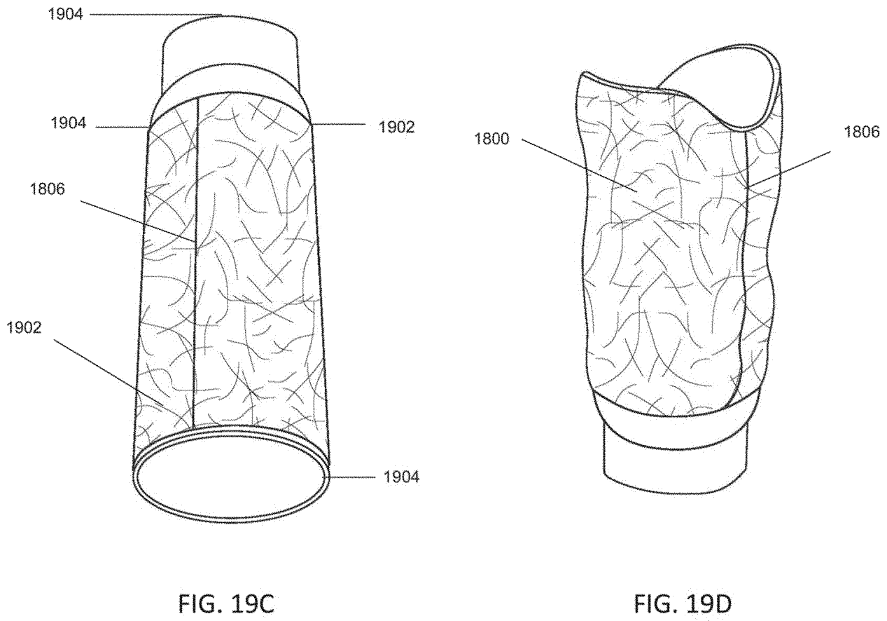

FIG. 19A is a perspective view of a prosthetic socket in accordance with an embodiment without an outer layer. FIG. 19B is a perspective view of the prosthetic socket of FIG. 19A in accordance with an outer layer partially arranged over a portion of the prosthetic socket. FIG. 19C is a perspective view of the prosthetic socket of FIG. 19A in accordance with the outer layer applied to the prosthetic socket. FIG. 19D is a perspective view of the prosthetic socket of FIG. 19A in accordance with the outer layer applied to the prosthetic socket in a trimmed configuration.

Referring to FIGS. 19A-19D, the prosthetic socket is generally represented with reference to number 1900. The prosthetic socket 1900 is used to join a residual limb to a prosthesis. For example, a lower calf portion of a residual limb can sit inside the prosthetic socket 1900 that attaches to a prosthetic mechanical lower leg to allow a person with a below or above the knee amputation to walk using the socket plus prosthesis. The prosthetic socket 1900 includes a conical cup 1902 and a lower portion 1904. In other embodiments, the prosthetic socket 1900 can include different components. The conical cup 1902 is in the shape of a hollow deep or elongated cup that is generally cylindrical in shape having a first end 1910 and second end 1912. The lower portion 1904 includes a first end 1906 and a second end 1908. The lower potion is configured also includes a cup region to receive a portion surrounding a portion of the conical cup 1902.

The conical cup 1902 is in the shape of a hollow deep or elongated cup that is generally cylindrical in shape having a first end 1910 and second end 1912. The conical cup 1902 is formed with an opening 1911 via which the residual limb can be inserted into the conical cup 1902. The conical cup 1902 is shapeable or moldable after being heated at a shaping temperature. The shaping temperature can be in the range from about 160° F. to about 305° F. and any sub-range within. In some embodiments, the conical cup 1902 has a pliability above a threshold pliability for a shaping time after being heated at the shaping temperature. The shaping time can be in the range of about 5 minutes to about 15 minutes, or any sub-range within. In one embodiment, during the shaping time, the conical cup 1906 can be stretched in various dimensions, e.g., circumferentially, over the residual limb or a model of the residual limb so that the conical cup 1920 is shaped to fit the residual limb.

In this embodiment, the conical cup 1902 does not include an outer layer 1800. Referring to FIG. 19B, the outer layer 1800 having an attached first side 1810 and second side 1812 is arranged over a portion of the conical cup 1902. Next, the outer layer 1800 is arranged over the conical cup between the first end 1910 and second end 1912. The outer layer 1800 is dimensioned with a length substantially equal to the length between the first end 1910 to the second end 1912 as shown in FIG. 19C.

Next the outer layer 1800 is heated with a heat source, e.g., oven, handheld heater, and the like. The outer layer is heated to a temperature in a range from about 160° F. to about 305° F. and any sub-range in between, in a preferred embodiment a temperature in a range from about 225° F. to 290° F., and in more preferred embodiment a temperature in a range from about 250° F. to about 285° F. During the heating step air is expanded and passes through one or more of the holes 1806. The heat also activates the heat activated adhesion material on the surface 1804 allowing the outer layer 1800 to be adhered to a surfaces of the conical cup 1902. Optionally and/or alternatively, the process can be repeated with another outer layer should a user get tired of the current graphic design or the outer layer becomes worn. Next the second end is trimmed as described herein to a predetermined geometry as desired by the clinician and described herein.

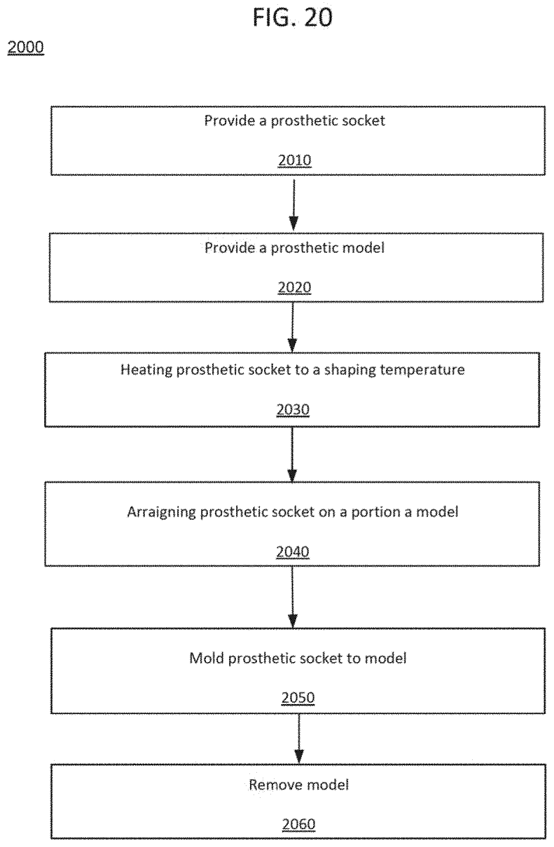

FIG. 20 is a flow chart illustrating a process of forming a prosthetic socket, in accordance with an embodiment.

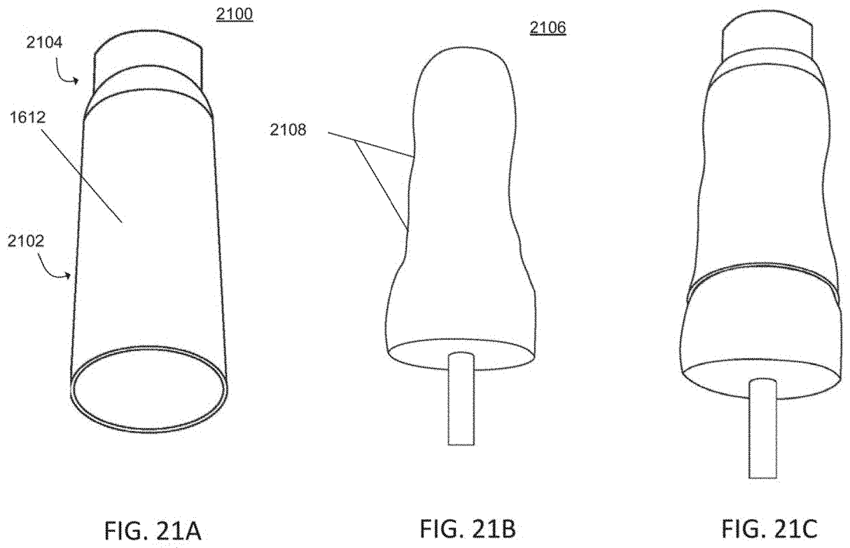

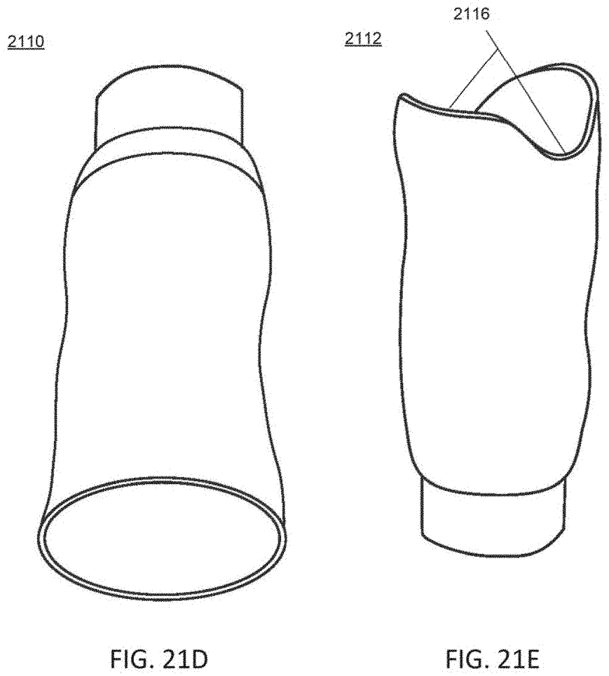

Referring to FIGS. 20 and 21A-C the prosthetic socket is used for joining a residual limb of a user with a prosthesis. The process may include different or additional steps than those described in conjunction with FIG. 20 in some embodiments or perform steps in different orders than the order described in conjunction with FIG. 20. A prosthetic socket 2100 is obtained (step 2010) as shown in FIG. 21A. The prosthetic socket 2100 includes a conical cup 2104 and lower portion 2104 as described herein. The conical cup 2102 becomes pliable after being heated at a shaping temperature, e.g., a temperature in the range from about 160° F. to about 305° F. The prosthetic socket 2100 can also include an outer layer and optionally a reinforcement layer as described herein. Optionally and/or alternatively, an outer layers 1700 is utilized with the conical cup 1902 and an outer layer 1706 is utilized with the lower portion 1904.

A prosthetic model 2106 is provided in step (step 2020). The prosthetic model 2106 as shown in FIG. 19B. The prosthetic model 2106 includes one or more contours 2108 that substantially mimic one or more contours of a user's limb. The construction of the prosthetic model 2106 from plaster or similar material is known in the art. The model 2106 is made based on at least a portion of a residual limb of a patient. For example, the model 2106 is a plaster model having a shape that matches the anatomical shape of the residual limb. Optionally and/or alternatively the prosthetic model 2106 has also been globally reduced. Global reduction is the process of hand filing, scraping and sanding of the plaster model to reduce the circumference in a global manner to make the resulting prosthetic socket more tightly fit the residual limb so as to bear weight in a more global manner reducing weight born by the residual bone end.

The prosthetic socket 2100 is heated (step 2030) to the shaping temperature as described herein. The outer layer 1612, conical cup 2102, and optional reinforcement layer become shapeable. The heated preformed prosthetic socket 2102 is arranged on a portion the model 2106 (step 2040) and molding (step 2050) the prosthetic socket to conform to one or more contours of the model to form the prosthetic socket as shown in FIG. 21C. The shaping or molding time can be in the range of five minutes to fifteen minutes, or any sub-range within. During the molding, the heated conical cup 2102 is stretched circumferentially directly over the model 2106 of the residual limb so that the conical cup 2102 is shaped to fit the residual limb by mimicking the one or more contours 2108 of the model 2106. Various techniques may be used to mold the prosthetic socket to the model e.g., hand shaping with gloves, vacuum forming, applying a compression sleeve or wrap, and the like. The model 2106, when cooled to room temperature and hardened is removed (step 2060) from the prosthetic socket as shown in FIG. 21D while leaving a molded prosthetic socket 2110 that substantially mimics one or more contours 2108 of the model 2106. In (step 2060) the molded prosthetic socket 2110 that is removed from the model 2106 can be trimmed and grinded to form a conformal opening 2116 as shown in FIG. 19E to form a molded and trimmed prosthetic socket 2112.

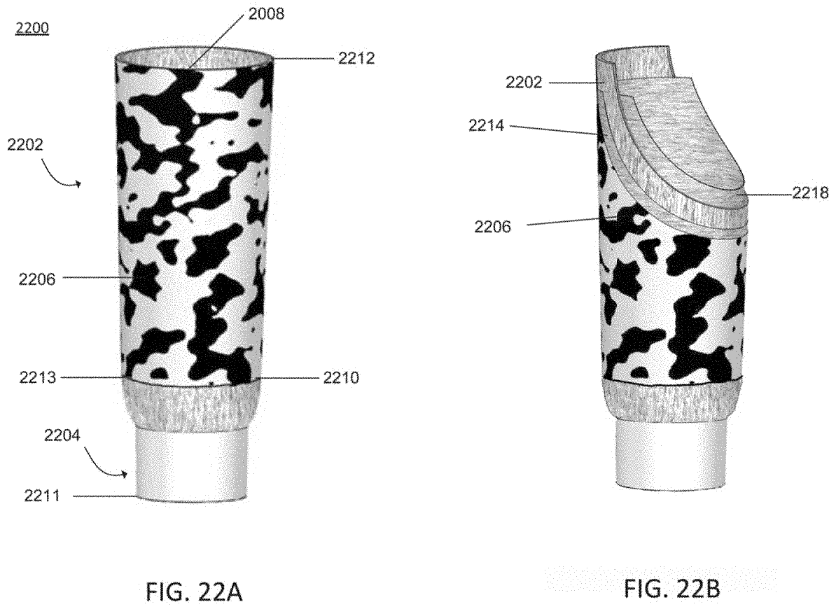

FIG. 22A is a perspective view of a prosthetic socket in accordance with an embodiment. FIG. 22B is a partial cross-sectional view of the prosthetic socket in FIG. 22A.

Referring to FIGS. 22A-22B, illustrates a prosthetic device 2200 that has been reinforced with one or more reinforcement inserts. The prosthetic socket 2200 is used to join a residual limb to a prosthesis. For example, a lower calf portion of a residual limb can sit inside the prosthetic socket 2200 that attaches to a prosthetic mechanical lower leg to allow a person with a below or above the knee amputation to walk using the socket plus prosthesis. The prosthetic socket 2200 includes a conical cup 2202 and a lower portion 2204. The conical cup 2202 has an opening 2208, a first end 2210 and a second end 2212. Optionally and/or alternatively, the conical cup 2202 has an outer layer 2206 as described herein.

The lower portion 2204 includes a first end 2211 and second end 2213. The conical cup 2202 is in a shape of a hollow deep or elongated cup that is generally cylindrical in shape having a first end 2210 and second end 2212. It has an outer surface and an inner surface. The conical cup 2202 is shapeable after being heated at a shaping temperature as described herein. The shaping temperature can be a temperature in the range of about 180° F. to about 305° F. and any sub-range within. In some embodiments, the conical cup 2202 has a pliability above a threshold pliability for a shaping time after being heated at the shaping temperature. The shaping time can be in the range of two minutes to ten minutes, or any sub-range within. During the shaping time, the conical cup 1202 can be stretched circumferentially directly over a residual limb or shaped over a model 1206 of the residual limb so that the conical cup 1202 is shaped to fit the residual limb.

In this embodiment a first insert 2214 and second insert 2218 can be utilized to increase a strength of the conical cup 2202, e.g., hoop strength. During an injection molding process thermoplastic flows from a first end of mold to a second end of the mold under pressure and heat. In such a case, the microscopic fibers tend to orient in the direction of flow, e.g., first end, e.g., bottom, to a second end, e.g., top. In such case, it is believed the conical cup is stronger in the top to bottom orientation than it is in the hoop direction because the fibers are much harder to separate longitudinally than they are across. The fibers may be any fibers or additives as described herein, e.g., carbon fibers, fiberglass fibers and the like.

In one embodiment, the conical cup 2202 includes additives, e.g., carbon fibers, oriented in a longitudinal direction from a first end 2210 to a second end 2212. The first insert 2214 is configured to have a strong hoop strength. In one embodiment, the first insert 2214 includes additives, e.g., one or more fibers in a hoop direction such as circumferentially. The second insert 2218 is configured to have a strong hoop strength with additives, e.g., one or more fibers in a hoop direction such as circumferentially.

In one embodiment, the first insert 2214 and second insert 2218 are made from an extruded sheet thermoplastic material. The extruded thermoplastic material includes fibers will see the fibers orient in the direction of the extrusion flow. If this sheet is then bent or formed into a conical tube, and seamed by using heat welding or adhesive, the carbon fibers can be oriented around the hoop direction. Only one insert can be used or both the first insert 2214 and second insert 2218 can be used during or after an injection molding process.

The first insert 2214 and second insert 2218 can have thickness from about 0.2 to about 1.5 mm or greater. The first insert 2214 and second insert 2218 a resized to have substantially the same length or smaller length from as the conical cup 2202 from the first end 2210 to the second end 2212.

In this embodiment, the lower portion 2204 can be injection molded from the same material or different material as conical cup 2202. Further, lower portion 2204, first insert 2214, second insert 2218, and outer layer 2206 can be injection molded with conical cup 2202, creating a unitary polymeric socket 2200. Stated another way, polymeric socket 2200 can include a one piece design where the conical cup 2202, and lower portion 2204 (including its components; outer layer 2206, first insert 2214, and second insert 2218) are all made jointly and simultaneously, and are essentially a single piece with the injection process.

Optionally and/or alternatively, the inserts can be injection molding with a side gate, blow molding or rotational molding which would see the carbon fibers more jumbled but still stronger in hoop strength compared to the socket.

Optionally and/or alternatively, the first insert 2214 and second insert 2218 could also be varied in thickness or have additional reinforcements adhered to it in specific spots, e.g., a carbon weave, or other composite. In one embodiment, the first insert 2214 and second insert 2218 are placed in the mold with one or more outer layer and the injection molding process.

In the present disclosure, the words “a” or “an” are to be taken to include both the singular and the plural. Conversely, any reference to plural items shall include, where appropriate, the singular.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size, and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

1. An outer layer for a prosthetic socket, the outer layer comprising:
an outer layer configured to be arranged around an outer circumference of a conical cup of the prosthetic socket and configured to be adhered to an outer surface of the conical cup of the prosthetic socket during a co-injection molding process, wherein the conical cup of the prosthetic socket comprises first end having an opening configured to receive at least a portion of a residual limb of a user and a second end being substantially closed,
wherein the outer layer comprises an outer layer thermoplastic material and, the outer layer has a thickness in range from of about 0.1 mm to about 1 mm,
wherein the outer layer thermoplastic material is malleable after being heated to a shaping temperature,
wherein the outer layer thermoplastic material is less malleable than a material of the conical cup of the prosthetic socket at the shaping temperature,
wherein at least a portion of the conical cup of the prosthetic socket is configured to be contained within the outer layer,
wherein the outer layer and the conical cup of the prosthetic socket are configured to be substantially simultaneously formed to one or more contours of a residual limb or one or more contours of a model of a residual limb at the shaping temperature, wherein during the forming the outer layer is less malleable than the conical cup of the prosthetic socket at the shaping temperature,
wherein the outer layer comprises a smoother outer surface as compared to an outer surface of the conical cup of the prosthetic socket at room temperature, and
wherein the outer layer has a higher abrasion resistance and scratch resistance as compared to an outer surface of the conical cup of the prosthetic socket at room temperature.

2. The outer layer of claim 1, wherein the outer layer and the conical cup of the prosthetic socket are co-injection molded.

3. The outer layer of claim 1, wherein the one or more layers is produced onto the prosthetic socket by a process, comprising the steps of:
forming a preliminary outer cup, the preliminary outer cup having a shape and size determined based on a size associated with at least the portion of the conical cup;
placing at least the portion of the conical cup within the preliminary outer cup; and
after at least the portion of the conical cup is placed within the preliminary outer cup, heating the preliminary outer cup at a temperature that the preliminary outer cup becomes malleable and adheres to the preliminary outer cup to form the outer layer on the conical cup.

4. The outer layer of claim 3, wherein the process further comprises the step of:
after at least the portion of the conical cup is placed within the preliminary outer cup, applying a vacuum pressure between the preliminary outer cup and the conical cup for forming the outer layer on the conical cup.

5. The outer layer of claim 1, wherein the abrasion resistant outer layer is substantially more resistant to being worn away by rubbing or friction than the conical cup.

6. The outer layer of claim 1, wherein the scratch resistant outer layer is configured substantially resistant to scratches.

7. The outer layer of claim 1, wherein the thickness is in a range from about 0.5 mm to about 1 mm.

8. The outer layer of claim 1, wherein the shaping temperature comprises a temperature in a range from about 180° F. to about 300° F.

9. The outer layer of claim 1, outer layer comprises an outside layer, one or more ink layers, and an adhesion layer.

10. The outer layer of claim 9, wherein the one or more ink layers is associated with a graphic pattern.

11. The outer layer of claim 9, wherein the one or more ink layers is associated with a logo.

12. The outer layer of claim 9, wherein the one or more ink layers is associated with a color.

13. The outer layer of claim 9, wherein the adhesive layer comprises a heat activated adhesive.

14. An outer layer for a prosthetic socket, the outer layer comprising:
a pre-dimensioned outer layer configured to be co-injected to form a prosthetic socket having a conical cup, a lower portion and the pre-dimensioned outer layer attached to an outer circumference of the conical cup,
wherein the pre-dimensioned outer layer comprises an inside surface, an opposite outer surface, a top, a bottom, a first side extending from the bottom to the top a first angle measured between the first side and the bottom, and second side opposite the first side extending from the bottom to the top at a second angle measured between the second side and the bottom, and a thickness in range from of about 0.1 mm to about 1 mm,
wherein the pre-dimensioned outer layer comprises a thermoplastic material that is malleable after being heated to a shaping temperature, and
wherein the pre-dimensioned outer layer and the conical cup of the prosthetic socket are configured to be substantially simultaneously formed to substantially mimic one or more contours of a residual limb or one or more contours of a model of a residual limb at the shaping temperature, and wherein when forming during the forming the pre-dimensioned outer layer to substantially mimic the one more contours of the residual limb or the one more contours of the model of a residual limb the pre-dimensioned outer layer is less malleable than the conical cup of the prosthetic socket at the shaping temperature.

15. The outer layer of claim 14, wherein the top comprises a top region extending from the first side to the second side and the top region in an arch type geometry.

16. The outer layer of claim 14, wherein the bottom comprises a bottom region extending from the first side to the second side in an arch type geometry.

17. The outer layer of claim 14, wherein the first angle is in a range from about 91 degrees to about 110 degrees.

18. The outer layer of claim 17, wherein the first angle is in a range from about 93 degrees to about 97 degrees relative to the bottom.

19. The outer layer of claim 14, wherein the second angle is in a range from about 91 degrees to about 110 degrees.

20. The outer layer of claim 19, wherein the second angle is in a range from about 93 degrees to about 97 degrees relative to the bottom.

21. The outer layer of claim 14, where the outer layer is substantially planar.

22. The outer layer of claim 14, wherein the pre-dimensioned outer layer further comprises one or more a graphic pattern, a logo, and a color on the outer surface.

23. The outer layer of claim 14, wherein the pre-dimensioned outer layer comprises an abrasion resistance surface.

24. The outer layer of claim 14, wherein the pre-dimensioned outer layer comprises a scratch resistance surface.

25. The outer layer of claim 14, wherein the inside surface comprises a heat activated adhesive.

26. The outer layer of claim 14, wherein the outer layer comprises a plurality of holes.

27. The outer layer of claim 14, wherein the plurality of holes are arranged in a grid pattern of about 0.2 inches.

28. The outer layer of claim 14, wherein each of the plurality of holes comprise a diameter in a range from about 0.001 inches to about 0.004 inches.

29. The outer layer of claim 14, wherein the first side is attached to the second side.

30. An outer layer for using in making a prosthetic socket, the outer layer comprising:
a substantially planar pre-dimensioned outer layer comprises a thermoplastic material configured to be co-injected molded to make the prosthetic socket, the prosthetic socket comprising a conical cup, a lower portion and the outer layer attached to and arranged around an outer circumference of the conical cup after the co-injected molded process,
wherein the outer layer comprises an inside surface, an opposite outside surface, a top, a bottom spaced apart from the top, a first side extending from the bottom to the top at a first angle measured between the bottom and the first side, and a second side opposite the first side extending from the bottom to the top at a second angle measured between the bottom and the second side, and a thickness in range from of about 0.1 mm to about 1 mm,
wherein the top comprises a top region extending between the first side to the second side and the top region comprises an arch type geometry,
wherein the bottom comprises a bottom region extending between the first side to the second side and the bottom region comprises an arch type geometry,
wherein the first angle is in a range from about 93 degrees to about 97 degrees,
wherein the second angle is in a range from about 93 degrees to about 97 degrees
wherein the thermoplastic material is malleable after being heated to a shaping temperature, and
wherein the outer layer and the conical cup of the prosthetic socket are configured to be substantially simultaneously formed to substantially mimic one or more contours of a residual limb or one or more contours of a model of a residual limb at the shaping temperature.

31. The outer layer of claim 26, wherein the shaping temperature is in a range from about 180° F. to about 300° F.

32-45. (canceled)
